(19) United States
(12) Patent Application Publication
(10) Pub. No.: US 2025/0289843 A1
(43) Pub. Date: Sep. 18, 2025

(54) PROCESS FOR PREPARATION OF TARGETING LIGANDS

(71) Applicant: Arrowhead Pharmaceuticals, Inc., Pasadena, CA (US)

(72) Inventors: Edward Cleator, Beerse (BE); Matthieu DORBEC, Beerse (BE); Brecht EGLE, Beerse (BE); Carl Arne Magnus ERIKSSON, Beerse (BE); William Marc MATON, Beerse (BE); Huibo SHENG, Beerse (BE); Koenraad WEGSTEEN, Beerse (BE)

(21) Appl. No.: 18/291,170

(22) PCT Filed: Jul. 22, 2022
(86) PCT No.: PCT/IB2022/056805

§ 371 Date: Jan. 22, 2024

Related U.S. Application Data
(60) Provisional application No. 63/224,969, filed on Jul. 23, 2021.

(51) Int. Cl. C07H 15/08; C07C 217/54; C07H 1/00

(52) U.S. Cl. CPC C07H 15/08; C07C 217/54; C07H 1/00; C07B 2200/13

(57) ABSTRACT

The present application provides synthetic processes for preparing N-acetyl-galactosamines (NAG) based compounds for targeted drug delivery. Also disclosed are Poly-NAG compounds, intermediates and targeting ligands, which are used and/or made by the methods described herein.

### FIELD OF THE INVENTION

The present application relates to compounds and methods for targeted drug delivery. Specifically, the invention provides a method or process of manufacturing targeting ligands for delivering therapeutic compounds to specific target sites.

### BACKGROUND OF THE INVENTION

Efficiently delivering a biologically active compound to a specific location in vivo, for example, to specific target cells, is important for therapeutic or diagnostic purposes. Traditional delivery systems such as oral ingestion or intravascular injection result in systemic distribution of compounds with low specificity for a target site (e.g., organ, tissue, cell type). In contrast, targeted delivery of a compound to a specific location concentrates the compound in the locations of interest while decreasing the concentration in other tissues. This can improve the compound's efficacy, and can also limit or potentially eliminate unintended consequences, such as side effects that arise from off-target interactions.

Many methods for delivering a compound to a desired location in vivo have been developed. One such method is to link or attach the compound to a targeting ligand comprised of one or more targeting group(s) or moiety(ies) which can serve to enhance the pharmacokinetic or bio-distribution properties of the compound to which it is linked. In particular, targeting moieties that are known to bind to the asiaglycoprotein receptor (ASGPR) are particularly useful in directing the delivery of therapeutic compounds to the liver. Asialoglycoprotein receptors are abundantly expressed on liver cells, including hepatocytes. Cell receptor targeting moieties that target ASGPR include galactose and galactose derivatives. In particular, clusters of galactose derivatives, including clusters comprised of two, three or four N-acetyl-galactosamines (GalNAc or NAG), can facilitate uptake of certain compounds in liver cells. For example, GalNAc clusters conjugated to therapeutic compounds have been shown to direct the composition to the liver, where the N-acetyl-galactosamine sugars are able to bind to the asialoglycoprotein receptors on the surface of the liver cell. The binding to an asialoglycoprotein receptor is believed to initiate receptor-mediated endocytosis, thereby facilitating entry of the compound into the interior of the cell.

Prior methods of synthesizing these targeting ligands generally result in the generation of synthetic intermediates with undesirable physical properties. For example, some process relies on intermediate compounds that are sticky oils are difficult to handle, rendering purification challenging and detrimentally affecting scalability. Therefore, a need exists in the art for improved processes in preparing NAG-based targeting ligands using methods based on intermediate compounds with improved physicochemical properties suitable for scaled-up manufacturing. The methods described herein address this unmet need.

### BRIEF SUMMARY OF THE INVENTION

In one aspect, provided herein is a method for preparing a poly-N-acetyl-galactosamines (poly-NAG) compound of Formula (X),

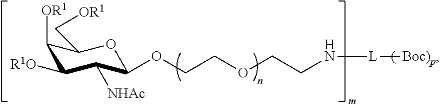

or a stereoisomer thereof, wherein R¹ is H or acetyl; n is an integer from 0 to 4; m is an integer from 3 to 6; p is an integer from 1 to 3; Boc is tert-butyloxycarbonyl and L is a branched linker comprising (1) m number of

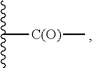

each of the wavy line indicates an attachment point to the remainder of the poly-NAG compound, or a stereoisomer thereof via nitrogen, and (2) p number of —NH—*, each of the (*) indicates an attachment point to the Boc group, wherein the method comprises a step of coupling a N-acetyl-galactosamine derivative of Formula (X-a),

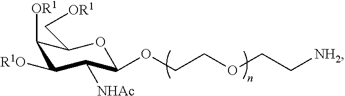

or a salt thereof, to a C₈-C₃₀ compound comprising (1) m number of carboxyl groups or ester groups, and (2) p number of Boc protected primary amine groups, to form the poly-NAG compound, or a stereoisomer thereof, and optionally where the compound of Formula (X-a) has been deprotected in situ in the presence of the C₈-C₃₀ compound, preferably where the compound of Formula (X-a) and the C₈-C₃₀ compound are present in a stoichiometric ratio so a salt forms between X-a and the C₈-C₃₀ compound. For example, the poly-NAG compound of Formula (X), or a stereoisomer thereof has a structure of

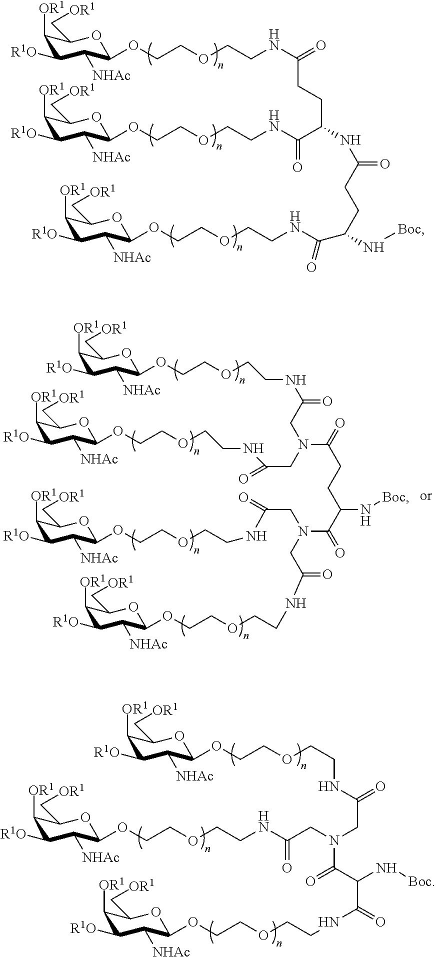

In some embodiments, the poly-NAG compound of Formula (X), or a stereoisomer thereof has a structure of

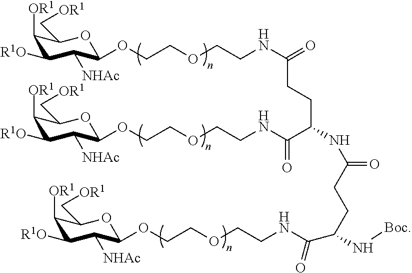

In some embodiments, the method further comprises a step of purifying the C₈-C₃₀ compound by crystallization prior to coupling to the N-acetyl-galactosamine derivative of Formula (X-a), or a salt thereof.

In some embodiments, the method further comprises a step of deprotecting the Bn groups of the C₈-C₃₀ compound. In some embodiments, the steps of deprotecting the Bn groups and coupling the N-acetyl-galactosamine derivative of Formula (X-a), or a salt thereof to the C₈-C₃₀ compound are performed together (e.g., without fully isolating a purified intermediate).

In some embodiments, the ester groups of the C₈-C₃₀ compound are benzyl (Bn) protected carboxyl groups. In some embodiments, the C₈-C₃₀ compound comprises m number of carboxyl groups or Bn protected carboxyl groups, and one Boc protected primary amine group. In some embodiments, the C₈-C₃₀ compound is

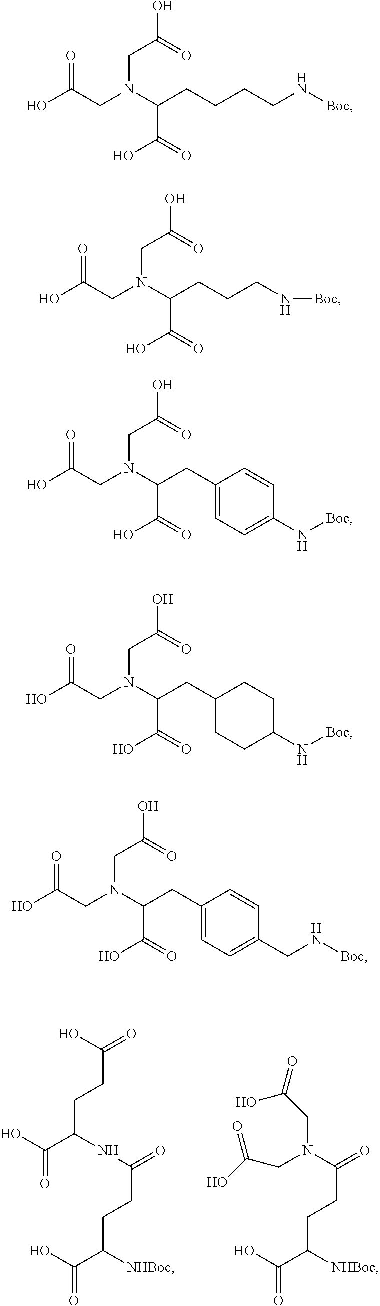

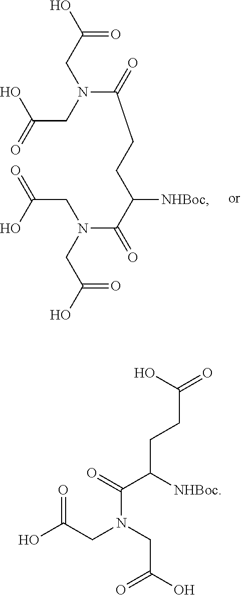 [SMILES: CC(C)(C)OC(=O)NC(CCC(=O)N(CC(=O)O)CC(=O)O)C(=O)N(CC(=O)O)CC(=O)O ; "or" ; CC(C)(C)OC(=O)NC(CCC(=O)O)C(=O)N(CC(=O)O)CC(=O)O]

For example, the C₈-C₃₀ compound is

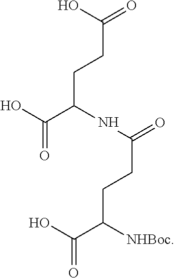 [SMILES: CC(C)(C)OC(=O)NC(CCC(=O)NC(CCC(=O)O)C(=O)O)C(=O)O]

In some embodiments, the C₈-C₃₀ compound is

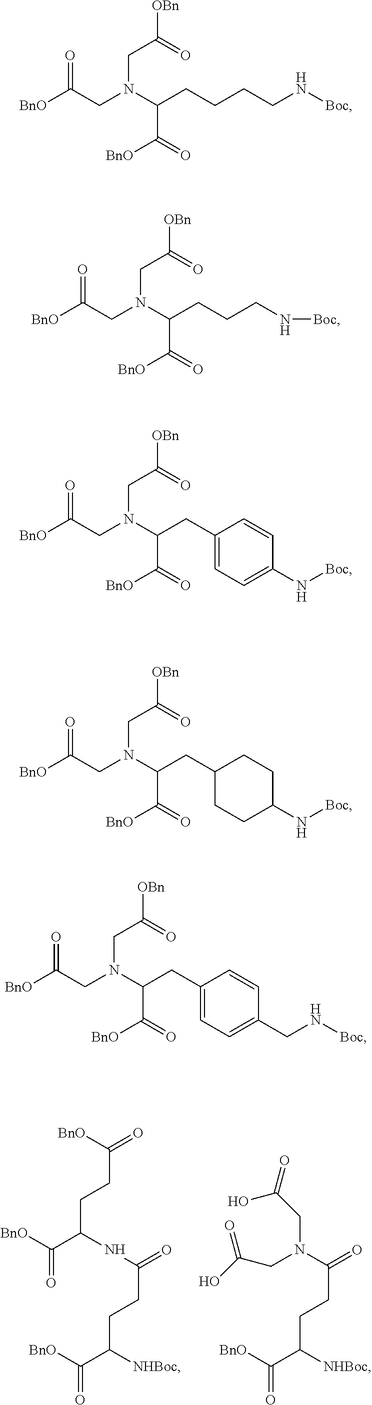

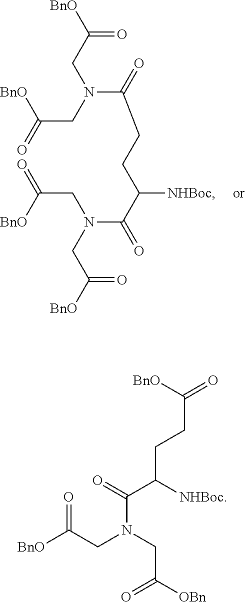 [SMILES: CC(C)(C)OC(=O)NC(CCC(=O)N(CC(=O)OCc1ccccc1)CC(=O)OCc1ccccc1)C(=O)N(CC(=O)OCc1ccccc1)CC(=O)OCc1ccccc1 ; CC(C)(C)OC(=O)NC(CCC(=O)OCc1ccccc1)C(=O)N(CC(=O)OCc1ccccc1)CC(=O)OCc1ccccc1]

For example, the C₈-C₃₀ compound is

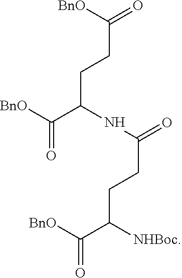 [SMILES: CC(C)(C)OC(=O)NC(CCC(=O)NC(CCC(=O)OCc1ccccc1)C(=O)OCc1ccccc1)C(=O)OCc1ccccc1]

In some embodiments, the method further comprises a step of deprotecting the Boc group of the poly-NAG compound of Formula (X), or a stereoisomer thereof, to obtain a primary amine compound of Formula (X-b),

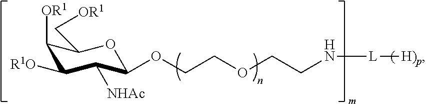

or a stereoisomer thereof. In some embodiments, the primary amine compound, or a stereoisomer thereof has a structure of

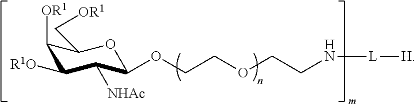

In some embodiments, the primary amine compound, or a stereoisomer thereof has a structure of

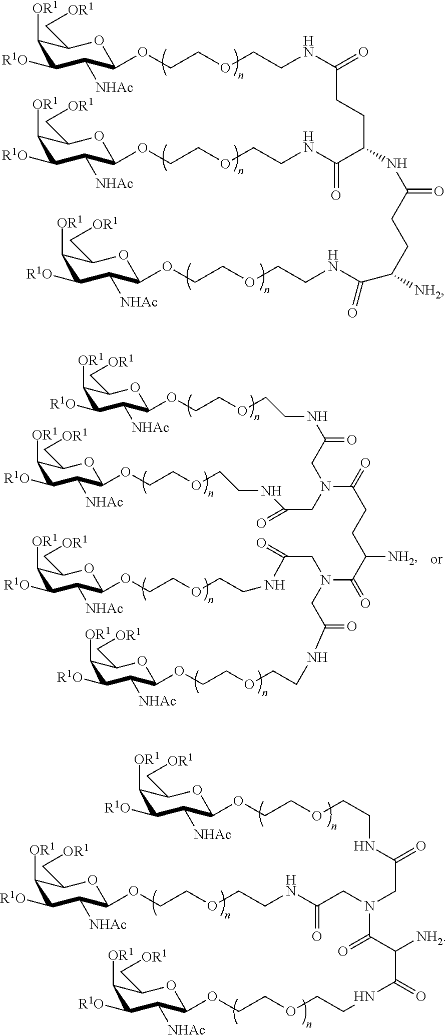

For example, the primary amine compound, or a stereoisomer thereof has a structure of

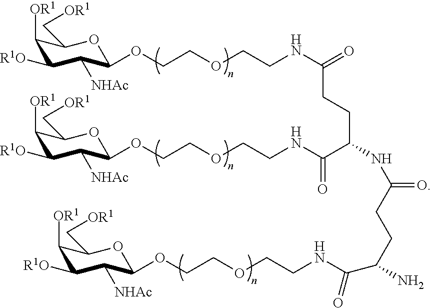

In some embodiments, the method further comprises a step of reacting the primary amine compound, or a stereoisomer thereof, through a condensation reaction with an acid of Formula (X-c),

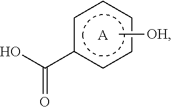

or a stereoisomer thereof, wherein ring A is cyclohexanyl or phenyl, to obtain a compound of Formula (X-d),

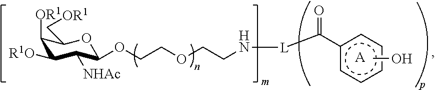

or a stereoisomer thereof. In some embodiments, the compound of Formula (X-d), or a stereoisomer thereof has a structure of

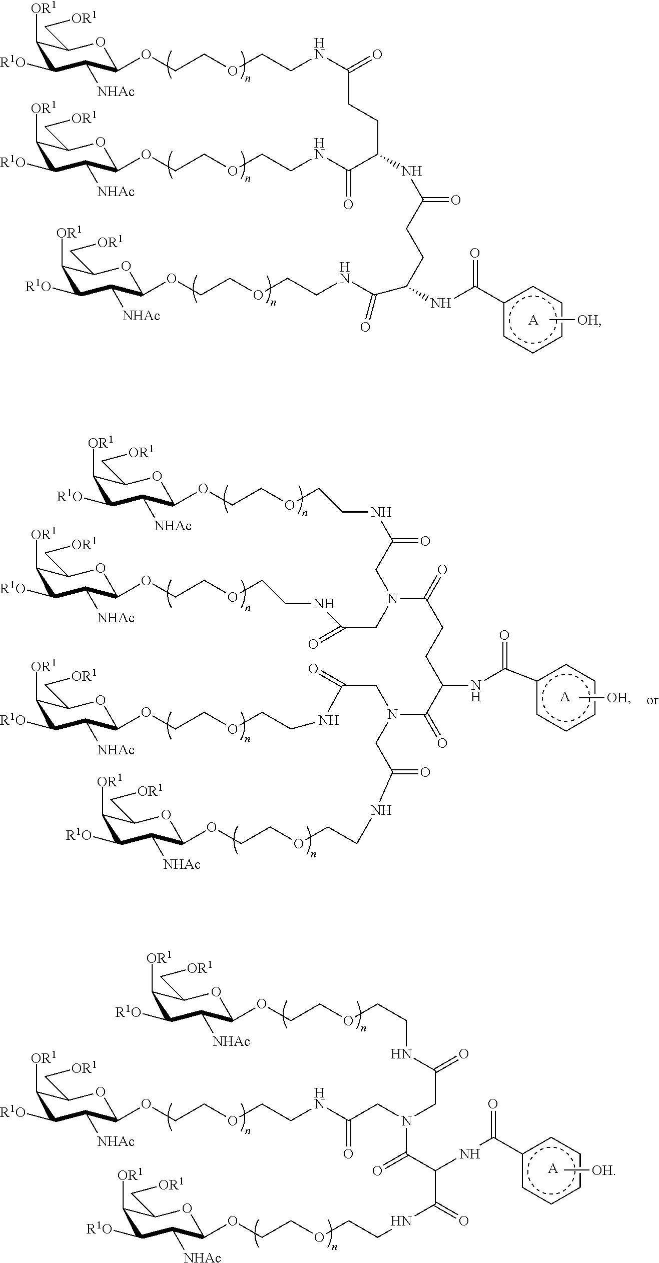

For example, the compound of Formula (X-d), or a stereoisomer thereof has a structure of

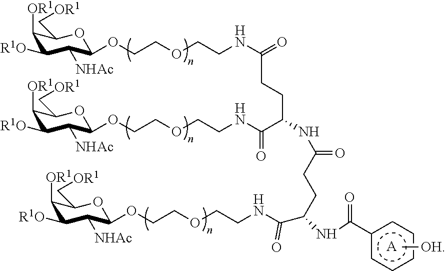

In some embodiments, the compound of Formula (X-d), or a stereoisomer thereof has a structure of

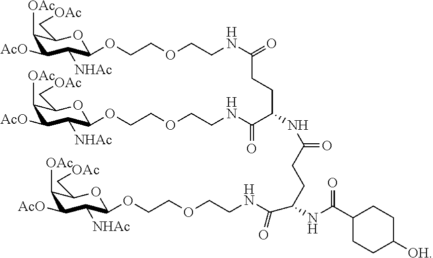

In some embodiments, the steps of deprotecting the Boc group and reacting with the acid of Formula (X-c), or a stereoisomer thereof, are performed together (e.g., without fully isolating a purified intermediate and/or performing the deprotection in the presence of the acid of Formula (X-c)).

In some embodiments, the method further comprises a step of linking the compound of Formula (X-d), or a stereoisomer thereof to a phosphoramidite reagent through a phosphitylation reaction forming a phosphoramidite compound of Formula (X-e),

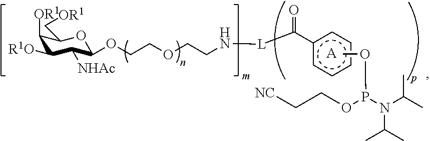

or a stereoisomer thereof. In some embodiments, the phosphoramidite compound of Formula (X-e), or a stereoisomer thereof has a structure of

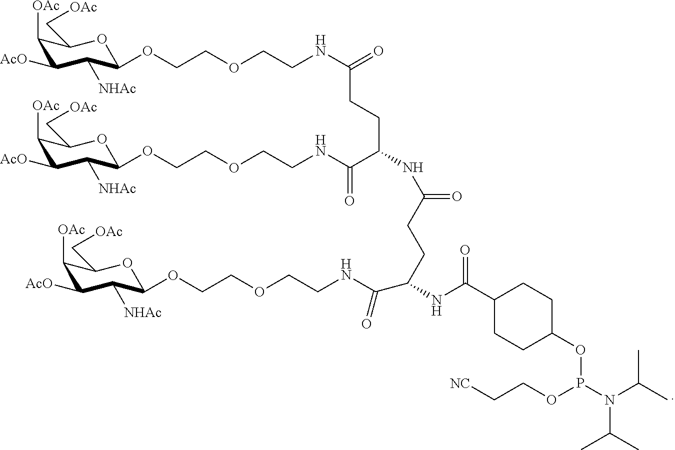

For example, the phosphoramidite compound of Formula (X-e), or a stereoisomer thereof has a structure of

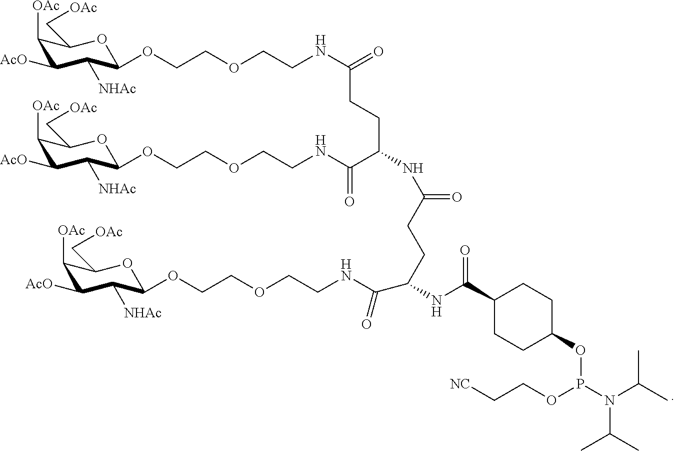

In some embodiments, the method further comprises a step of covalently attaching a therapeutic agent to the phosphoramidite compound of Formula (X-e), or a stereoisomer thereof. In some embodiments, the therapeutic agent is an expression-inhibiting oligomeric compound. In some embodiments, the expression-inhibiting oligomeric compound is an RNAi agent.

In some embodiments, the C₈-C₃₀ compound further comprises one or more amide bonds. The method can further comprise a step of reacting a first compound comprising one or more carboxyl groups with a second compound comprising one or more primary amine groups via an amide reaction to produce the C₈-C₃₀ compound.

In some embodiments, the ester groups of the C₈-C₃₀ compound are Bn protected carboxyl groups. In some embodiments, the method further comprises a step of deprotecting the Bn groups of the C₈-C₃₀ compound. In some embodiments, the steps of reacting of the first and second compounds, and deprotecting the Bn groups are performed together (e.g., without fully isolating a purified intermediate) prior to coupling to the N-acetyl-galactosamine derivative of Formula (X-a), or a salt thereof.

In some embodiments, the method further comprises a step of deprotecting a benzyloxycarbonyl (Cbz) group of a protected N-acetyl-galactosamine derivative of Formula (X-f),

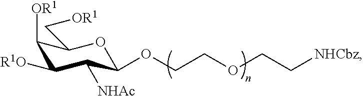

or a salt thereof, to obtain the N-acetyl-galactosamine derivative of Formula (X-a), or a salt thereof, optionally where the protected N-acetyl-galactosamine derivative of Formula (X-f) is deprotected in the presence of the C₈-C₃₀ compound, such as the tri-acid of Formula (4), thereby forming a C₈-C₃₀ compound acid salt of the N-acetyl-galactosamine derivative of Formula (X-a). In some embodiments, the step of deprotecting the Cbz group is conducted in flow chemistry. In some embodiments, the salt is a trifluoroacetic acid (TFA) salt. In some embodiments, the steps of deprotecting the Cbz group, and coupling the N-acetyl-galactosamine derivative of Formula (X-a), or a salt thereof to the C₈-C₃₀ compound are performed together (e.g., without fully isolating a purified intermediate and/or performing the deprotection in the presence of the C₈-C₃₀ compound). In some embodiments, the ester groups of the C₈-C₃₀ compound are Bn protected carboxyl groups. In some embodiments, the method further comprises deprotecting the Bn groups of the C₈-C₃₀ compound. In some embodiments, the steps of deprotecting the Cbz group, deprotecting the Bn groups, and coupling the N-acetyl-galactosamine derivative of Formula (X-a), or a salt thereof to the C₈-C₃₀ compound are performed together (e.g., without fully isolating a purified intermediate and/or performing the deprotection in the presence of the C₈-C₃₀ compound).

In some embodiments, n is 3 or 4.

In some embodiments, p is 1.

In some embodiments, R¹ is acetyl and n is 1.

Also provided is a method for preparing a phosphoramidite compound of Formula (I)

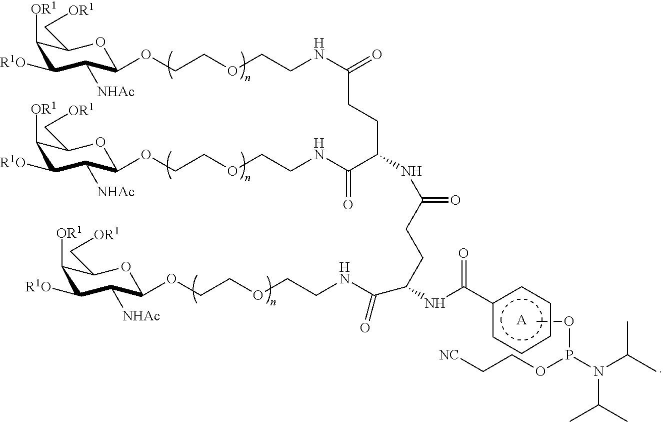

or a stereoisomer thereof, wherein R¹ is H or acetyl, n is an integer from 0 to 4, and ring A is cyclohexanyl or phenyl, comprising:
(i) reacting a compound of Formula (1),

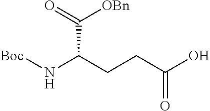 [SMILES: CC(C)(C)OC(=O)N[C@@H](CCC(=O)O)C(=O)OCc1ccccc1]

or a stereoisomer thereof, with a compound of Formula (2),

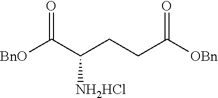

or a stereoisomer thereof, to obtain a compound of Formula (3),

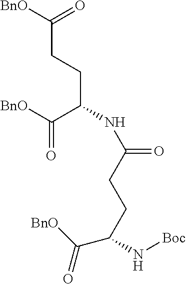 [SMILES: CC(C)(C)OC(=O)N[C@@H](CCC(=O)N[C@@H](CCC(=O)OCc1ccccc1)C(=O)OCc1ccccc1)C(=O)OCc1ccccc1]

or a stereoisomer thereof;
(ii) deprotecting three benzyl (Bn) groups of the compound of Formula (3) or a stereoisomer thereof, to obtain a tri-acid compound of Formula (4),

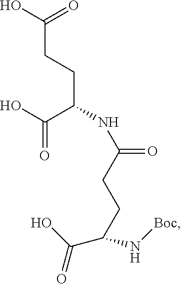 [SMILES: CC(C)(C)OC(=O)N[C@@H](CCC(=O)N[C@@H](CCC(=O)O)C(=O)O)C(=O)O]

or a stereoisomer thereof;
(iii) coupling a N-acetyl-galactosamine derivative of Formula (X-a), or a salt thereof,

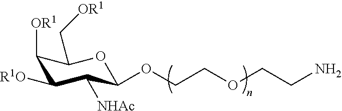

to the tri-acid compound of Formula (4) or a stereoisomer thereof, to form a tri-N-acetyl-galactosamines (tri-NAG) compound of Formula (5),

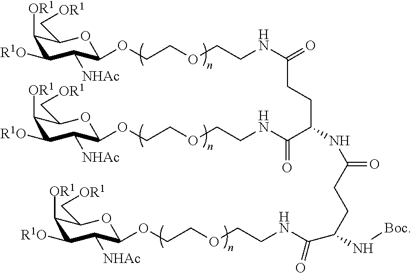

or a stereoisomer thereof;
(iv) deprotecting a tert-butyloxycarbonyl (Boc) group of the tri-N-acetyl-galactosamines (tri-NAG) compound of Formula (5) or a stereoisomer thereof, to obtain a tri-N-acetyl-galactosamines (tri-NAG) compound of Formula (6),

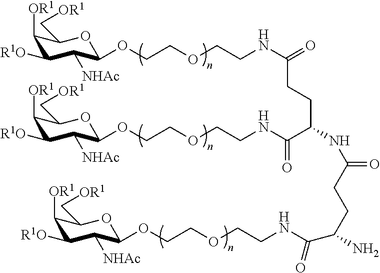

or a stereoisomer thereof;
(v) reacting the tri-N-acetyl-galactosamines (tri-NAG) compound of Formula (6) or a stereoisomer thereof, with an acid of Formula (X-c)

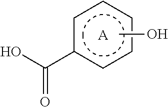

or a stereoisomer thereof, through a condensation reaction between the primary amine group of the tri-N-acetyl-galactosamines (tri-NAG) compound of Formula (6) and the carboxyl group of the acid of Formula (X-c) or a stereoisomer thereof, to obtain a tri-N-acetyl-galactosamines (tri-NAG) compound of Formula (7) or a stereoisomer thereof

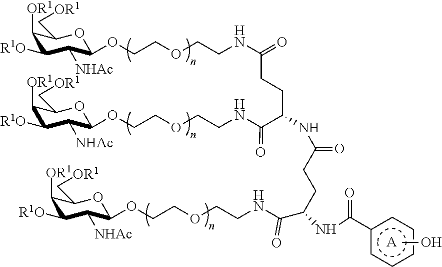

(vi) linking the tri-N-acetyl-galactosamines (tri-NAG) compound of Formula (7) or a stereoisomer thereof, to a phosphorus atom of a phosphoramidite reagent through a phosphitylation reaction forming the phosphoramidite compound of Formula (I) or a stereoisomer thereof.

In some embodiments, the steps (i) and (ii) are performed together (e.g., without fully isolating a purified intermediate and/or in the presence of the reactant from the other step). In some embodiments, the steps (ii) and (iii) are performed together (e.g., without fully isolating a purified intermediate and/or in the presence of the reactant from the other step). In some embodiments, the steps (iv) and (v) are performed together (e.g., without fully isolating a purified intermediate and/or in the presence of the reactant from the other step).

In some embodiments, the tri-acid compound of Formula (4), or a stereoisomer thereof, in the step (II) is purified by crystallization. For example, the crystallization is performed in a solvent, which is selected from the group consisting of acetonitrile (MeCN), tetrahydrofuran (THF), isopropylacetate (IPAc), water, isopropyl alcohol (IPA), or any combination thereof.

In some embodiments, coupling in step (iii) is performed in the presence of an agent. The agent can be 1-Ethyl-3-(3-dimethylaminopropyl)carbodiimide (EDCI), 1-2-(1H-benzotriazol-1-yl)-1,1,3,3-tetramethyluronium hexafluorophosphate (HBTU), hydroxybenzotriazole (HOBt) or any combination thereof. In certain embodiments, the agent is EDCI.

In some embodiments, the condensation reaction in the step (v) is conducted in the presence of an agent. The agent can be 1-Ethyl-3-(3-dimethylaminopropyl)carbodiimide (EDCI), hydroxybenzotriazole (HOBt), 2-Hydroxypyridine-N-oxide (HOPO) or combination thereof. In certain embodiments, the agent is EDCI.

In some embodiments, the phosphitylation reaction in the step (vi) is conducted in the presence of an agent. The agent can be tetrazole, ethylthiotetrazole (ETT), benzylthiotetrazole (BTT), N-methylimidazole (NMI) or dicyanoimidazole (DCI), and combination thereof. In some embodiments, the agent is tetrazole.

In some embodiments, the molar ratio of the phosphoramidite reagent to the tri-NAG compound of Formula (7) or a stereoisomer thereof, in the step (vi) is about 1.5.

In some embodiments, the method further comprises a step of deprotecting a benzyloxycarbonyl (Cbz) group of a protected N-acetyl-galactosamine derivative of Formula (8),

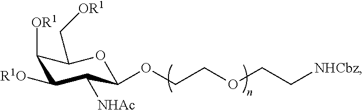

to obtain the N-acetyl-galactosamine derivative of Formula (X-a), or a salt thereof. In some embodiments, the salt is a trifluoroacetic acid (TFA) salt. In some embodiments, the step (iii) and the step of deprotecting the benzyloxycarbonyl (Cbz) group are performed together (e.g., without fully isolating a purified intermediate and/or in the presence of the reactant from the other step). In some embodiments, the steps (ii) and (iii), and the step of deprotecting the benzyloxycarbonyl (Cbz) group are performed together (e.g., without fully isolating a purified intermediate and/or in the presence of the reactant from the other step).

In another aspect, provided is a phosphoramidite compound of Formula (I), or a stereoisomer thereof prepared by the method described herein. Also provided is a therapeutic compound prepared by covalently attaching a therapeutic agent via the phosphorus atom to the phosphoramidite compound of Formula (I), or a stereoisomer thereof.

Also provided is a tri-N-acetyl-galactosamines (tri-NAG) compound having a structure of

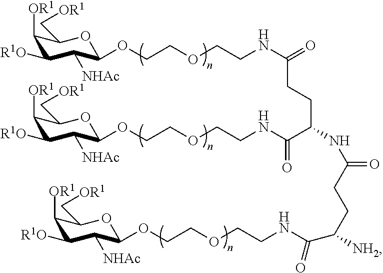

or a stereoisomer thereof, wherein R¹ is H or acetyl, and n is an integer from 0 to 4.

Also provided is a tert-butyloxycarbonyl (Boc) group protected tri-N-acetyl-galactosamines (tri-NAG) compound having a structure of

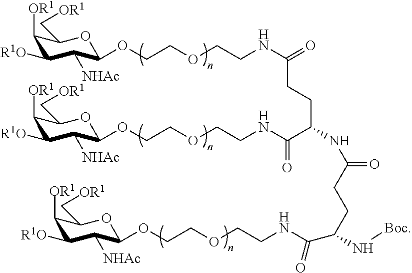

or a stereoisomer thereof, wherein R¹ is H or acetyl, and n is an integer from 0 to 4.

In addition, provided are an intermediate having a structure of

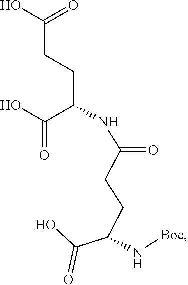 [SMILES: CC(C)(C)OC(=O)N[C@@H](CCC(=O)N[C@@H](CCC(=O)O)C(=O)O)C(=O)O]

or a stereoisomer thereof, and an intermediate having a structure of

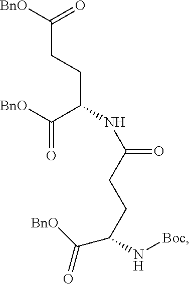 [SMILES: CC(C)(C)OC(=O)N[C@@H](CCC(=O)N[C@@H](CCC(=O)OCc1ccccc1)C(=O)OCc1ccccc1)C(=O)OCc1ccccc1]

or a stereoisomer thereof. In some embodiments, the intermediates are in a crystalline form.

### DETAILED DESCRIPTION OF THE INVENTION

Described herein are processes or methods of manufacturing poly-NAG (e.g., tri-NAG) targeting ligands. In some embodiments, the targeting ligands are linked to compounds, such as therapeutic or diagnostic compounds. The targeting ligands can be used to target compounds, such as expression-inhibiting oligomeric compounds, to a desired location of a target nucleic acid or target gene.

The present application describes targeting ligands comprising essentially one or more targeting moieties connected via a linker to a therapeutic compound. In some embodiments, the targeting ligands are phosphoramidite compounds containing targeting moieties based on N-acetyl-galactosamine (NAG), as shown below. The synthesis method as described herein can provide a more efficient approach for making and scaling up the targeting ligands. It requires fewer reaction steps and provides high yield synthesis without the need of purifying several intermediates, which are suitable for the scale up process.

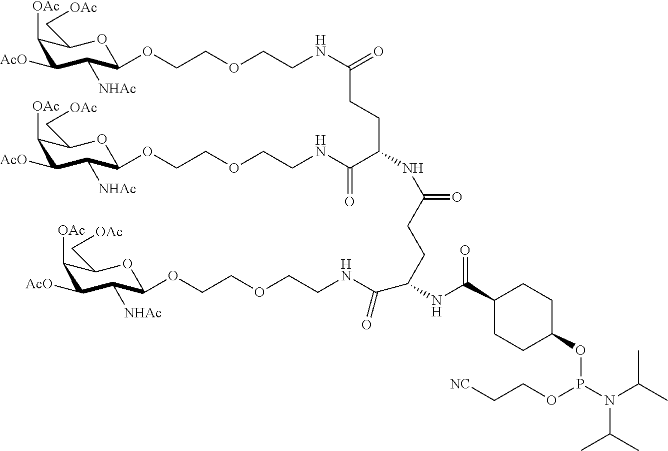

These targeting ligands can be conjugated to therapeutic compounds and used in a variety of applications. One class of therapeutic compounds that can be targeted using targeting ligands are oligomeric compounds. Oligomeric compounds that include nucleotide sequences at least partially complementary to a target nucleic acid have been shown to alter the function and activity of the target both in vitro and in vivo. When delivered to a cell containing a target nucleic acid (such as mRNA), oligomeric compounds have been shown to modulate the expression of the target resulting in altered transcription or translation of the target nucleic acid. In certain instances, the oligomeric compound can reduce the expression of the gene by inhibiting the nucleic acid target and/or triggering the degradation of the target nucleic acid.

Compositions comprising a targeting ligand conjugated to expression-inhibiting oligomeric compounds are capable of mediating expression of target nucleic acid sequences in target cells. These compositions may be helpful in the treatment of diseases or conditions that respond to inhibition of gene expression in a specific cell or tissue.

Previous methods of synthesizing these NAG-containing targeting ligands rely on the generation of poly(acid) intermediate compounds containing a carboxybenzyl (Cbz)-protected amine group. These intermediates were sticky oils that were difficult to handle and limited the scalability of the overall synthetic procedure. The present invention is based, in part, on the discovery that intermediate compounds containing tert-butyloxycarbonyl (Boc) groups in place of the Cbz groups used in the previously disclosed methods results in intermediates with improved physical properties that streamline the overall synthetic process, improving scalability and potential for manufacturing. In some embodiments, intermediates are crystalline solids rather than sticky oils.

Unless otherwise stated or implied by context, terms that are used herein have the meanings defined below. Unless otherwise contraindicated or implied, e.g., by including mutually exclusive elements or options, in those definitions and throughout this specification, the terms “a” and “an” mean one or more and the term “or” means and/or where permitted by context. Thus, as presented in the specification and the appended claims, the singular forms “a,” “an,” and “the” include plural referents unless the context clearly dictates otherwise.

As used herein, the term “linked” when referring to the connection between two molecules means that the two molecules are joined by a covalent bond.

As used herein, an oligomeric compound is a nucleotide sequence containing about 10-50 nucleotides or nucleotide base pairs. In some embodiments, an oligomeric compound has a nucleobase sequence that is at least partially complementary to a coding sequence in an expressed target nucleic acid or target gene within a cell. In some embodiments, the oligomeric compounds, upon delivery to a cell expressing a gene, are able to inhibit the expression of the underlying gene, and are referred to herein as expression-inhibiting oligomeric compounds. The gene expression can be inhibited in vitro or in vivo. Oligomeric compounds include, but are not limited to: oligonucleotides, single-stranded oligonucleotides, single-stranded antisense oligonucleotides, short interfering RNAs (siRNAs), double-strand RNA (dsRNA), micro RNAs (miRNAs), short hairpin RNAs (shRNA), ribozymes, interfering RNA molecules, and dicer substrates.

In some embodiments, a salt is a pharmaceutically acceptable salt. A pharmaceutically acceptable salt of a given compound, for instance a compound of Formula 1, refers to salts that retain the biological effectiveness and properties of a given compound, and which are not biologically or otherwise undesirable. In some embodiments, a pharmaceutically acceptable salt of a given compound, for instance a compound of Formula 1, refers to that a salt form which is generally regarded as safe and suitable for use without undue toxicity, irritation, allergic response, and the like, commensurate with a reasonable benefit/risk ratio. Exemplary pharmaceutically acceptable salts include acid addition salts formed with inorganic acids such as hydrochloric acid, hydrobromic acid, sulfuric acid, nitric acid, phosphoric acid, and the like; or formed with organic acids such as acetic acid, benzenesulfonic acid, benzoic acid, camphorsulfonic acid, citric acid, ethanesulfonic acid, fumaric acid, glucoheptonic acid, gluconic acid, lactic acid, maleic acid, malonic acid, mandelic acid, methanesulfonic acid, 2-napththalenesulfonic acid, oleic acid, palmitic acid, propionic acid, stearic acid, succinic acid, tartaric acid, p-toluenesulfonic acid, trimethyl acetic acid, trifluoroacetic acid (TFA) and trifluoromethanesulfonic acid (TfOH), and the like, and salts formed when an acidic proton present in the parent compound is replaced by either a metal ion, e.g., an alkali metal ion, an alkaline earth ion, or an aluminum ion; or coordinates with an organic base such as diethanolamine, triethanolamine, N-methylglucamine and the like. Also included in this definition are ammonium and substituted or quaternized ammonium salts. In some embodiments, the salt includes a counterion derived from a “C₈-C₃₀ compound” as described herein, such as an “C₈-C₃₀ compound” having one or more (e.g., 1, 2, 3 or 4) free carboxylic acid moieties.

As used herein, unless specifically identified in a structure as having a particular conformation, for each structure in which asymmetric centers are present and thus give rise to enantiomers, diastereomers, or other stereoisomeric configurations, each structure disclosed herein is intended to present all such possible isomers, including their optically pure and racemic forms. Fr example, the structures disclosed herein are intended to cover mixtures of diastereomers as well as single stereoisomers.

A number of embodiments of the invention are described below followed by a more detailed discussion of the components, e.g., groups, reagents, and steps that are useful in the process of the present invention. Any of the specified embodiments for the components of the processes can apply to each and every aspect of the invention as described herein, or they may relate to a single aspect. The selected embodiments may be combined together in any combination appropriate for preparing a poly-NAG-containing targeting ligand.

In one aspect, provided herein is a method for preparing a poly-N-acetyl-galactosamines (poly-NAG) compound of Formula (X),

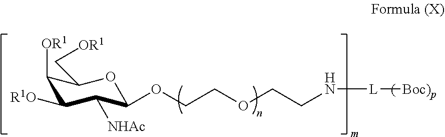

or a salt or stereoisomer thereof, wherein:
R¹ is H or acetyl;
n is an integer from 0 to 4;
m is an integer from 3 to 6;
p is an integer from 1 to 3;
Boc is tert-butyloxycarbonyl and
   L is a branched linker comprising (1) m number of

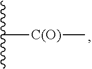

each of the wavy line indicates an attachment point to the remainder of the poly-NAG compound, or a stereoisomer thereof via nitrogen, and (2) p number of —NH—*, each of the (*) indicates an attachment point to the Boc group.

In some embodiments, R¹ is H or acetyl. In some embodiments, R¹ is H. In some embodiments, R¹ is acetyl.

In some embodiments, m is 3, 4, 5, or 6. In some embodiments, m is 3. In some embodiments, m is 4. In some embodiments, m is 5. In some embodiments, m is 6.

In some embodiments, n is 1, 2, 3, or 4. In some embodiments, n is 1. In some embodiments, n is 2. In some embodiments, n is 3. In some embodiments, n is 4.

In some embodiments, p is 1, 2, or 3. In some embodiments, p is 1. In some embodiments, p is 2. In some embodiments, p is 3.

In some embodiments, R¹ is acetyl and n is 1. In some embodiments, R¹ is acetyl, n is 1, and m is 3. In some embodiments, R¹ is acetyl, n is 1, m is 3, and p is 1.

In some embodiments, the poly-NAG compound of Formula (X), or a salt or stereoisomer thereof has a structure of

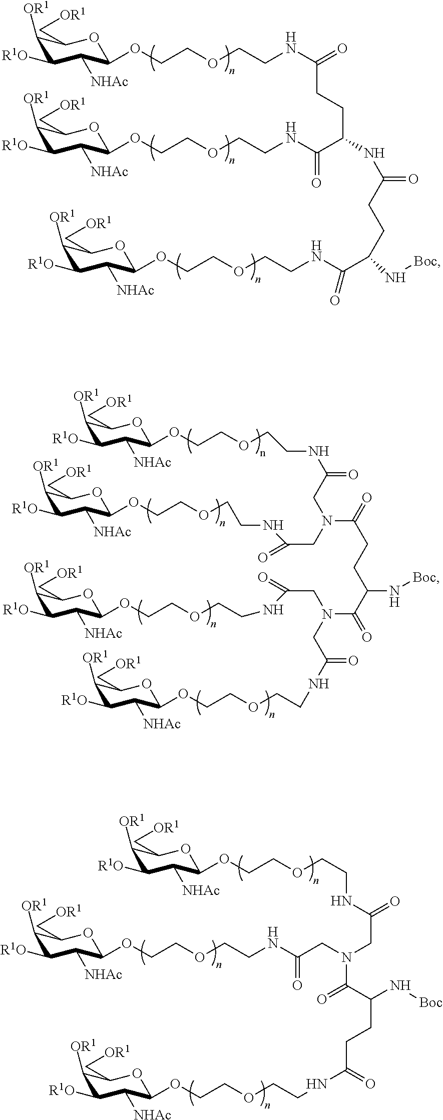

or a salt thereof. In some embodiments, the poly-NAG compound of Formula (X), or a salt or stereoisomer thereof has a structure of

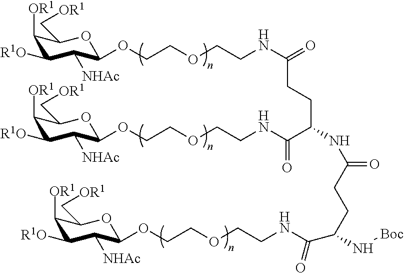

or a salt thereof.

In some embodiments, the method for preparing a poly-NAG compound of Formula (X) comprises Step 1: coupling a N-acetyl-galactosamine derivative of Formula (X-a),

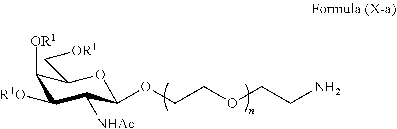

or a salt thereof, to a C₈-C₃₀ compound comprising (1) m number of carboxyl groups or ester groups, and (2) p number of Boc protected primary amine groups, to form the poly-NAG compound, or a stereoisomer thereof. In some embodiments, the compound of Formula (X-a) is in a salt form with a C₈-C₃₀ compound, such as the tri-acid of Formula (4).

As used herein, the phrase “C₈-C₃₀ compound” refers to a moiety that is based on a backbone-carbon chain containing between 8 and 30 carbon atoms. The C₈-C₃₀ compounds disclosed herein can be of any group which permits attachment of one or more NAG compounds of Formula (X-a) and further includes a protected amine group. In some embodiments, the C₈-C₃₀ compounds disclosed herein comprise one carboxylic acid moiety (or ester thereof) for each desired linkage to a NAG compound of Formula (X-a), and at least one amine group (or protected derivative thereof). In some embodiments, the C₈-C₃₀ compound further comprises one or more amide bonds. In some embodiments, the C₈-C₃₀ compound comprises a number of carboxyl groups or Bn protected carboxyl groups, and one Boc protected primary amine group.

In some embodiments, the C₈-C₃₀ compound is

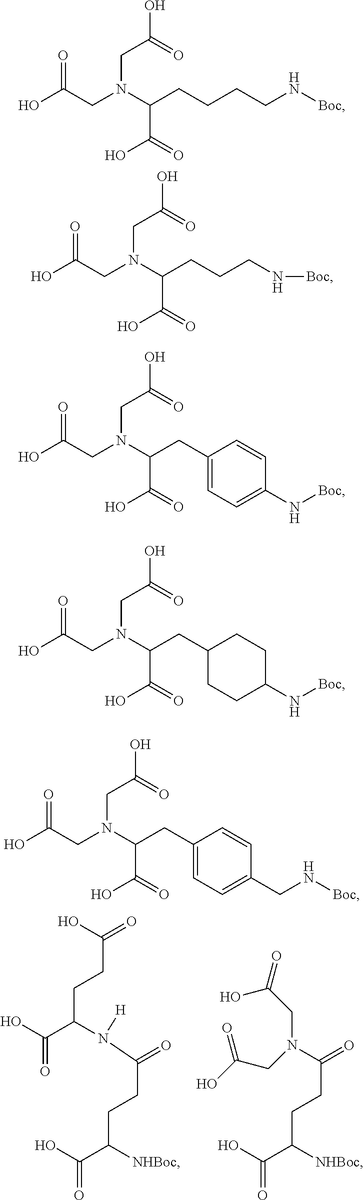

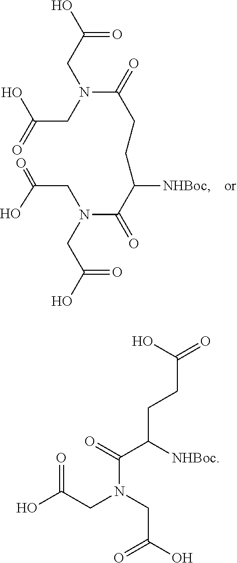 [SMILES: CC(C)(C)OC(=O)NC(CCC(=O)N(CC(=O)O)CC(=O)O)C(=O)N(CC(=O)O)CC(=O)O ; CC(C)(C)OC(=O)NC(CCC(=O)O)C(=O)N(CC(=O)O)CC(=O)O]

In some embodiments, the C₈-C₃₀ compound is

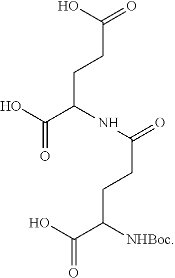 [SMILES: CC(C)(C)OC(=O)NC(CCC(=O)NC(CCC(=O)O)C(=O)O)C(=O)O]

In some embodiments, the C₈-C₃₀ compound is

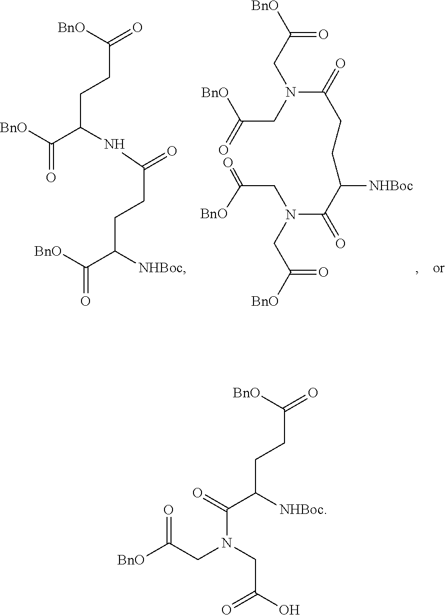 [SMILES: CC(C)(C)OC(=O)NC(CCC(=O)NC(CCC(=O)OCc1ccccc1)C(=O)OCc1ccccc1)C(=O)OCc1ccccc1 ; CC(C)(C)OC(=O)NC(CCC(=O)N(CC(=O)OCc1ccccc1)CC(=O)OCc1ccccc1)C(=O)N(CC(=O)OCc1ccccc1)CC(=O)OCc1ccccc1 ; CC(C)(C)OC(=O)NC(CCC(=O)OCc1ccccc1)C(=O)N(CC(=O)O)CC(=O)OCc1ccccc1]

In some embodiments, the C₈-C₃₀ compound is

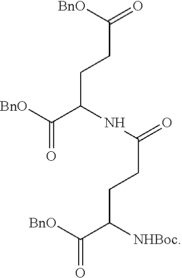 [SMILES: CC(C)(C)OC(=O)NC(CCC(=O)NC(CCC(=O)OCc1ccccc1)C(=O)OCc1ccccc1)C(=O)OCc1ccccc1]

In some embodiments, Step 1 is performed in the presence of a base. In some embodiments, the base is pyridine, 4,-dimethyl aminopyridine (DMAP), triethylamine, isopropylethylamine, imidazole, 1,4-diazabicyclo[2.2. 2]octane (DABCO), 1,8-Diazabicyclo[5.4.0]undec-7-ene (DBU), 2,6-lutidine, N-methylimidazole, N-methylmorpholine (NMM), or N,N-diisopropylethylamine (DIPEA). In some embodiments, the base is NMM. In some embodiments, the base is DIPEA.

In some embodiments, Step 1 is performed in the presence of an agent. In some embodiments, the agent is 1-ethyl-3-(3-dimethylaminopropyl)carbodiimide (EDCI), isobutyl chloroformate, hydroxybenzotriazole (HOBt), 2-(1H-benzotriazol-1-yl)-1,1,3,3-tetramethyluronium hexauorophosphate (HBTU), propylphosphonic anhydride, 1,1′-carbonyldiimidazole (CDI), 2-hydroxypyridine-N-oxide (HOPO) or a mixture thereof. In some embodiments, the agent is EDCI. In some embodiments, the agent is HOBt. In some embodiments, the agents are EDCI and HOBt. In some embodiments, the agents are HBTU and HOBt.

In some embodiments, Step 1 is performed in solution with some, or all of the reaction components dissolved and/or suspended in one or more solvents. In some embodiments, the solvent is acetonitrile, acetone, dichloromethane dimethylformamide, dimethylsulfoxide, N-methyl-2-pyrrolidone, 1,4-dioxane, tetrahydrofuran, IPA (isopropanol, 2-propanol), or a mixture thereof. In some embodiments, the solvent is tetrahydrofuran. In some embodiments, the solvent is dichloromethane. In some embodiments, the solvent is dimethylformamide. In some embodiments, the solvent is a mixture of dichloromethane and dimethylformamide. In some embodiments, the solvent is isopropyl acetate.

In some embodiments, the N-acetyl-galactosamine derivative of Formula (X-a), or a salt thereof, that is used in Step 1 is obtained by deprotecting a benzyloxycarbonyl (Cbz) group of a protected N-acetyl-galactosamine derivative of Formula (X-f),

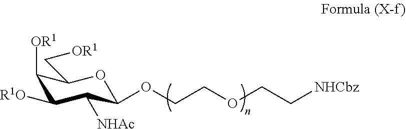

or a salt thereof. In some embodiments, the compound of Formula (X-f) is deprotected in the presence of a C₈-C₃₀ compound (such as the tri-acid of Formula (4)). In some embodiments, the compound of Formula (X-f) and the C₈-C₃₀ compound, such as the tri-acid of Formula (4), are in a molar ratio of about 1:1 to about 3:1, e.g., about 1:1, 1.5:1, 2:1, 2.5:1 or 3:1). In some embodiments, the deprotected compound of Formula (X-f) forms a salt with the C₈-C₃₀ compound, such as the tri-acid of Formula (4).

In some embodiments, the salt is a trifluoroacetic acid (TFA) salt. In some embodiments, the step of deprotecting the Cbz group is conducted in flow chemistry. In some embodiments, the step of deprotecting the Cbz group is performed under reductive conditions. In some embodiments, the step of deprotecting the Cbz group is performed under reductive conditions using a catalyst and hydrogen gas. In some embodiments, the catalyst is Pd/C. In some embodiments, the step of deprotecting the Cbz group is performed under reductive conditions using a catalyst and hydrogen gas, and an acid additive. In some embodiments, the acid additive is toluenesulfonic acid, methanesulfonic acid, hydrochloric acid, sulfuric acid, or trifluoroacetic acid. In some embodiments, the acid additive is trifluoroacetic acid.

In some embodiments, the step of deprotecting the Cbz group is performed under hydrogen gas. In some embodiments, the pressure of hydrogen gas is about atmospheric pressure, about 10-15 psi, or about 40-50 psi.

In some embodiments, the step of deprotecting the Cbz group is performed in solution with some, or all of the reaction components dissolved and/or suspended in one or more solvents. In some embodiments, the solvent is tetrahydrofuran, dichloromethane, or isopropanol. In some embodiments, the solvent is a mixture of tetrahydrofuran, dichloromethane, or isopropanol. In some embodiments, the solvent is tetrahydrofuran. In some embodiments, the solvent is isopropanol.

In some embodiments, the ester groups of the C₈-C₃₀ compound are benzyl (Bn) protected carboxyl groups. In some embodiments, the method for preparing a compound of Formula (X) further comprises the step of deprotecting the Bn groups of the C₈-C₃₀ compound.

In some embodiments, the step of deprotecting the Bn groups is performed under reductive conditions. In some embodiments, the step of deprotecting the Bn groups is performed under reductive conditions using a catalyst and hydrogen gas. In some embodiments, the catalyst is Pd/C. In some embodiments, the step of deprotecting the Bn groups is performed under hydrogen gas. In some embodiments, the pressure of hydrogen gas is atmospheric pressure, 10-15 psi, or 40-50 psi.

In some embodiments, the step of deprotecting the Bn groups is performed in solution with some, or all of the reaction components dissolved and/or suspended in one or more solvents. In some embodiments, the solvent is tetrahydrofuran.

In some embodiments, the steps of deprotecting the Cbz group, and Step 1, i.e., coupling the N-acetyl-galactosamine derivative of Formula (X-a), or a salt thereof to the C₈-C₃₀ compound, are performed together (e.g., without fully isolating a purified intermediate and/or deprotecting in the presence of the C₈-C₃₀ compound). In some embodiments, the steps of deprotecting the Cbz group, deprotecting the Bn groups, and Step 1, i.e., coupling the N-acetyl-galactosamine derivative of Formula (X-a), or a salt thereof to the C₈-C₃₀ compound, are performed together (e.g., without fully isolating a purified intermediate and/or deprotecting in the presence of the C₈-C₃₀ compound). In some embodiments, the compound of Formula (X-a) is in a salt form with a C₈-C₃₀ compound, such as the tri-acid of Formula (4).In some embodiments, suitable solvents for these combined steps include dichloromethane, isopropyl alcohol, tetrahydrofuran, or mixtures thereof.

In some embodiments, the method for preparing a compound of Formula (X) further comprises purifying the C₈-C₃₀ compound by crystallization prior to coupling to the N-acetyl-galactosamine derivative of Formula (X-a), or a salt thereof. In some embodiments, the crystallization is performed in a solvent, which is selected from the group consisting of acetonitrile (MeCN), tetrahydrofuran (THF), isopropylacetate (IPAc), water, isopropyl alcohol (IPA), and any combination thereof.

In some embodiments, the method for preparing a poly-NAG compound of Formula (X) further comprises Step 2: deprotecting the Boc group of the poly-NAG compound of Formula (X), or a stereoisomer thereof, to obtain a primary amine compound of Formula (X-b),

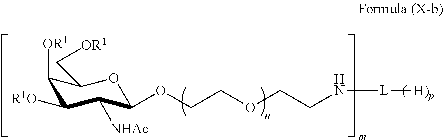

or a salt or stereoisomer thereof. In some embodiments, the compound of Formula (X-a) is in a salt form with an acid, such as TFA and/or TfOH.

In some embodiments, the primary amine compound, or a salt or stereoisomer thereof has a structure of

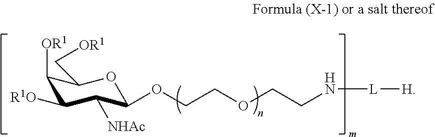

In some embodiments, the primary amine compound, or a salt or stereoisomer thereof has a structure of

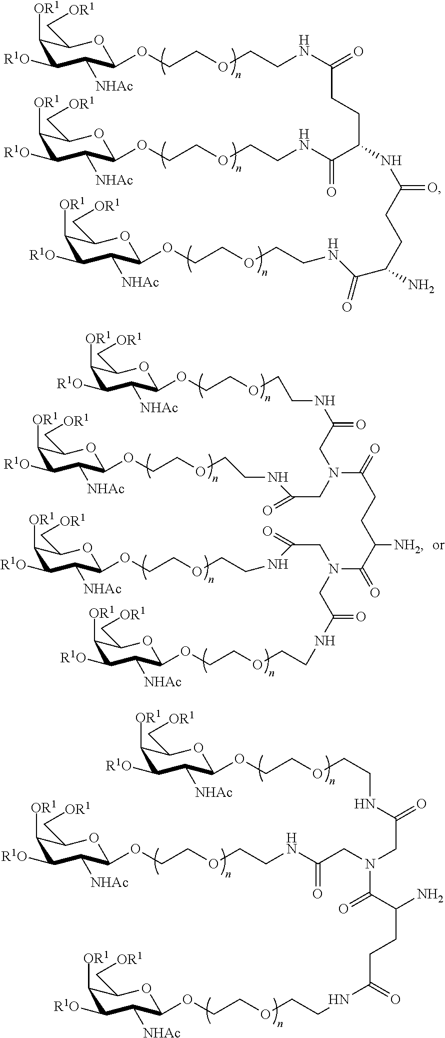

or a salt thereof. In some embodiments, the primary amine compound, or a salt or stereoisomer thereof has a structure of

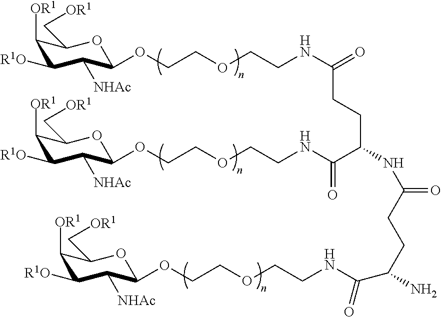

or a salt thereof.

In some embodiments, Step 2 is performed using a Lewis acid. In some embodiments, the Lewis acid is a trialkylsilyl trihalalkylsufonate. In some embodiments, the Lewis acid is trimethylsilyl trifluoromethanesulfonate (TMSOTf). In some embodiments, the Lewis acid also functions as a silylating agent. In some embodiments, Step 2 is performed with a dehydrating agent. In some embodiments, the dehydrating agent is an acetamide derivative. In some embodiments, the dehydrating agent is N,O-bis(trimethylsilyl)acetamide (BSA). In some embodiments, the dehydrating agent also functions as a silylating agent. In some embodiments, Step 2 is performed with both TMSOTf and BSA. In some embodiments, Step 2 is performed without BSA and/or with only TMSOTf. In some embodiments, Step 2 is performed using azeotropic distillation.

In some embodiments, Step 2 is performed in solution with some, or all of the reaction components dissolved and/or suspended in one or more solvents. In some embodiments, the solvent comprises acetonitrile, acetone, dichloromethane dimethylformamide, dimethylsulfoxide, N-methyl-2-pyrrolidone, 1,4-dioxane, and/or tetrahydrofuran. In some embodiments, the solvent comprises acetonitrile. In some embodiments, the solvent comprises acetonitrile and at least one additional solvent (e.g., dichloromethane).

In some embodiments, the method for preparing a poly-NAG compound of Formula (X) further comprises Step 3: reacting the primary amine compound, or a salt or stereoisomer thereof, through a condensation reaction with an acid of Formula (X-c),

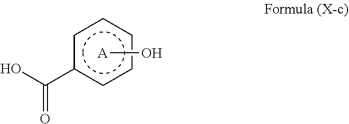

or a salt or stereoisomer thereof, wherein ring A is cyclohexanyl or phenyl, to obtain a compound of Formula (X-d),

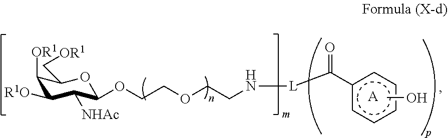

or a salt or stereoisomer thereof.

In some embodiments, the compound of Formula (X-d), or a salt or stereoisomer thereof, has a structure of

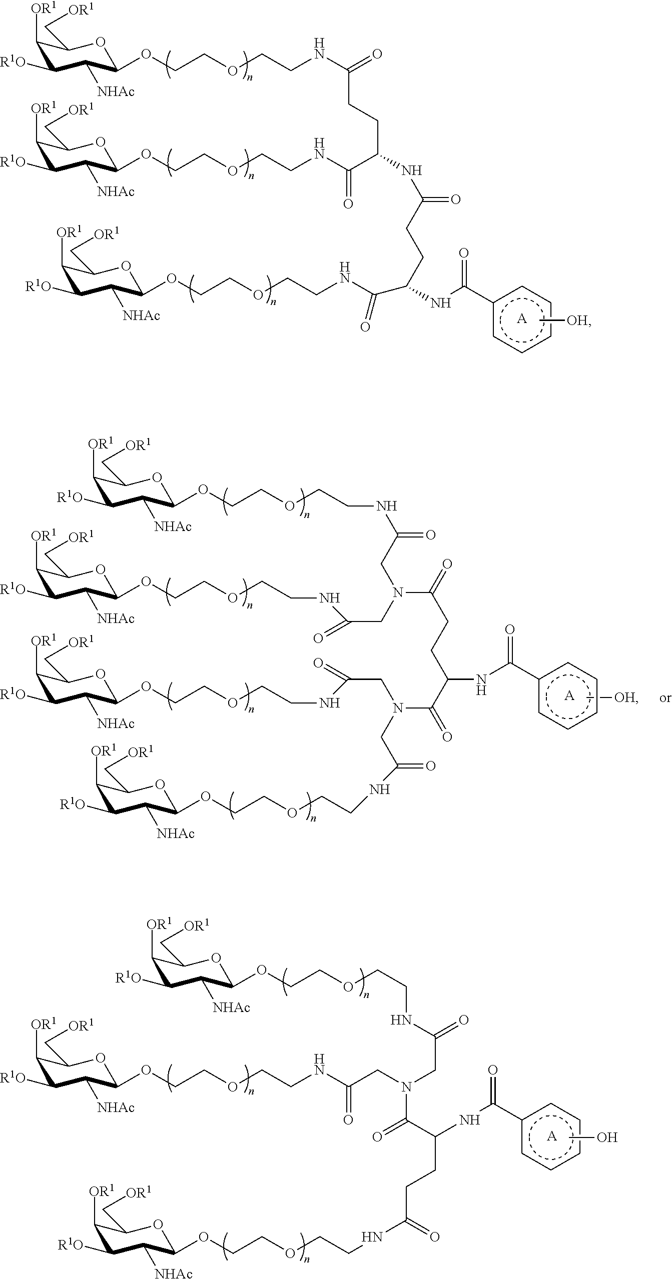

or a salt thereof.

In some embodiments, the compound of Formula (X-d), or a salt or stereoisomer thereof, has a structure of

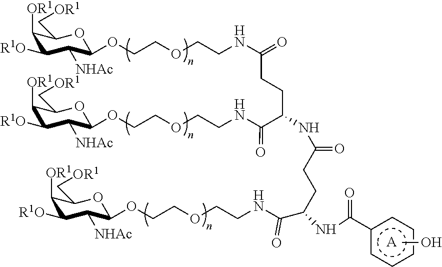

or a salt thereof.

In some embodiments, the acid of Formula (X-c) has a structure of

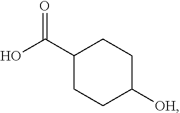

or a salt or stereoisomer thereof.

In some embodiments, the compound of Formula (X-d), or a salt or stereoisomer thereof, has a structure of

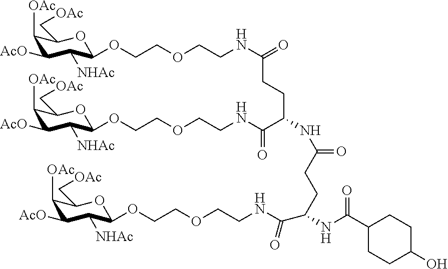

or a salt thereof.

In some embodiments, the compound of Formula (X-d) has a structure of

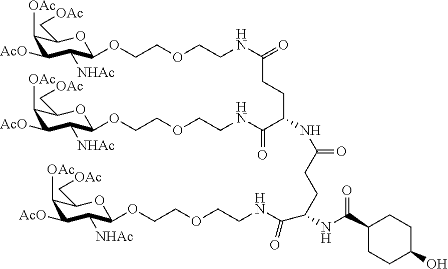

or a salt thereof.

In some embodiments, Step 3 is performed in the presence of a base. In some embodiments, the base is an amine base, such as pyridine, DMAP, triethylamine, isopropylethylamine, imidazole, DABCO, DBU, 2,6-lutidine, N-methylimidazole, NMM, or DIPEA, or a combination thereof. In some embodiments, the base comprises DIPEA.

In some embodiments, Step 3 is performed in the presence of an agent. In some embodiments, the agent is EDCI, isobutyl chloroformate, HOBt, HBTU, propylphosphonic anhydride, CDI, HOPO, or a mixture thereof. In some embodiments, the agent is EDCI. In some embodiments, the agent is HOBt. In some embodiments, the agent is HOPO. In some embodiments, the agents are EDCI and HOBt. In some embodiments, the agents are EDCI and HOPO.

In some embodiments, Step 3 is performed in solution with some, or all of the reaction components dissolved and/or suspended in one or more solvents. In some embodiments, the solvent comprises acetonitrile, acetone, dichloromethane dimethylformamide, dimethylsulfoxide, N-methyl-2-pyrrolidone, 1,4-dioxane, tetrahydrofuran, or a mixture thereof. In some embodiments, the solvent comprises acetonitrile. In some embodiments, the solvent comprises dichloromethane.

In some embodiments, the method for preparing a poly-NAG compound of Formula (X) further comprises a process where the Steps 2 and 3, i.e., deprotecting the Boc group and reacting with the acid of Formula (X-c), are performed together (e.g., without fully isolating a purified intermediate and/or in combination with reaction components from the other step). In some embodiments, Step 2 is performed as described herein, then water is added to the reaction mixture and Step 3 is subsequently performed as described herein. In some embodiments, the amount of water that is added is about 1 to about 3 molar equivalent(s) with respect to the poly-NAG compound of Formula (X) (e.g., about 1, 1.5, 2, 2.5, or 3 molar equivalent(s)).

In some embodiments, the method for preparing a poly-NAG compound of Formula (X) further comprises Step 4: linking the compound of Formula (X-d), or a salt or stereoisomer thereof to a phosphoramidite reagent through a phosphitylation reaction forming a phosphoramidite compound of Formula (X-e),

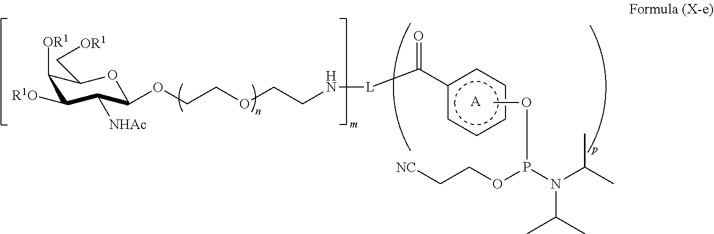

or a salt or stereoisomer thereof.

In some embodiments, the phosphoramidite compound of Formula (X-e) has a structure of

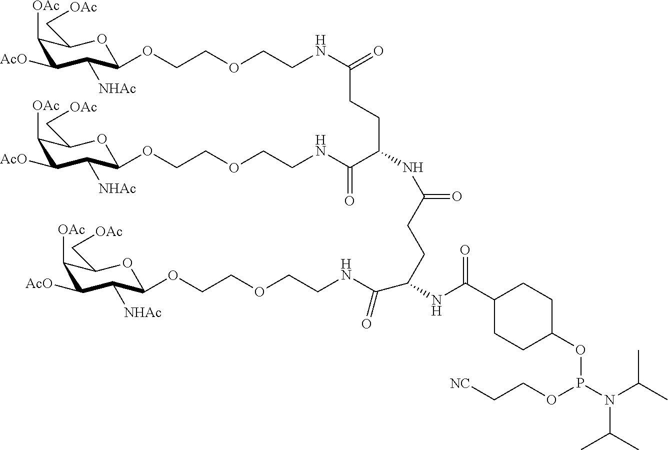

or a salt thereof. In some embodiments, the phosphoramidite compound of Formula (X-e) has a structure of

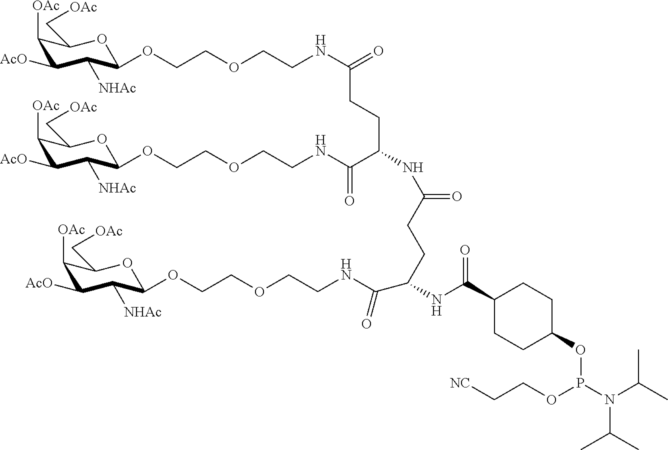

or a salt thereof.

In some embodiments of the phosphitylation reaction, the phosphoramidite reagent is

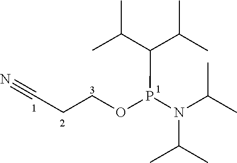 [SMILES: CC(C)C(C(C)C)P(OCCC#N)N(C(C)C)C(C)C]

3-((bis(diisopropylamino)phosphaneyl)oxy)propanenitrile or

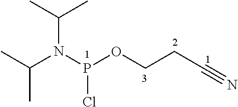

3-((chloro(diisopropylamino)phosphaneyl)oxy)propanenitrile.

In some embodiments, Step 4 is conducted in the presence of an agent. In some embodiments, the activating agent is tetrazole, ethylthiotetrazole (ETT), benzylthiotetrazole (BTT), N-methylimidazole (NMI) or dicyanoimidazole (DCI), and combination thereof. In some embodiments, the agent is tetrazole.

In some embodiments, Step 4 is conducted under reaction conditions that minimize exposure of the reaction components to water or moisture. In some embodiments, the concentration of water in the reaction is less than 100 ppm. In some embodiments, the technique of limiting exposure to water involves purifying the solvent or solvents used in the reaction. In some embodiments, the purification involves azeotropic distillation of the solvent prior to use. In some embodiments, the solvent is dichloromethane that has undergone azeotropic distillation. In certain embodiments, the result of Step 4 is that the water content of the solution of X-d in solvent (e.g., dichloromethane) is reduced. In certain embodiments the source of water in the solution of compound of Formula (X-d) is from water present in the solid form of the compound of Formula (X-d).

In some embodiments of Step 4, the molar ratio of the phosphoramidite reagent to the tri-NAG compound of Formula (7) or a salt or stereoisomer thereof, is about 1.5.

In some embodiments, the phosphoramidite compound of Formula (X-e) or a salt or stereoisomer thereof is dried and stored below room temperature.

In some embodiments, Step 4 is performed in solution with some, or all of the reaction components dissolved and/or suspended in one or more solvents. In some embodiments, the solvent is acetonitrile, acetone, dichloromethane dimethylformamide, dimethylsulfoxide, N-methyl-2-pyrrolidone, 1,4-dioxane, or tetrahydrofuran. In some embodiments, the solvent is dichloromethane.

A therapeutic agent can be covalently attached to the phosphoramidite compound of Formula (X-e), or a salt or stereoisomer thereof. In some embodiments, the therapeutic agent is an expression-inhibiting oligomeric compound. In some embodiments, the expression-inducing oligomeric compound is an RNAi agent.

In some embodiments, a method is provided for preparing a compound of Formula (X) that comprises performing Steps 1 through 6. In some embodiments, the method comprises performing Steps 1 through 5. In some embodiments, the method comprises performing Steps 1 through 4. In some embodiments, the method comprises performing Steps 1 through 3. In some embodiments, the method comprises performing Steps 1 and 2. In some embodiments, the method comprises performing Steps 2 through 6. In some embodiments, the method comprises performing Steps 2 through 5. In some embodiments, the method comprises performing Steps 2 through 4. In some embodiments, the method comprises performing Steps 2 and 3. In some embodiments, the method comprises performing Steps 3 through 6. In some embodiments, the method comprises performing Steps 3 through 5. In some embodiments, the method comprises performing Steps 3 and 4. In some embodiments, the method comprises performing Steps 4 through 6. In some embodiments, the method comprises performing Steps 5 and 6. In some embodiments, any two or more of Steps 1, 2, 3, 4, 5, and 6 may be performed together (e.g., without fully isolating a purified intermediate and/or in the presence of one or more reaction components from another step).

In some embodiments, a process is provided for preparing a compound of Formula (I):

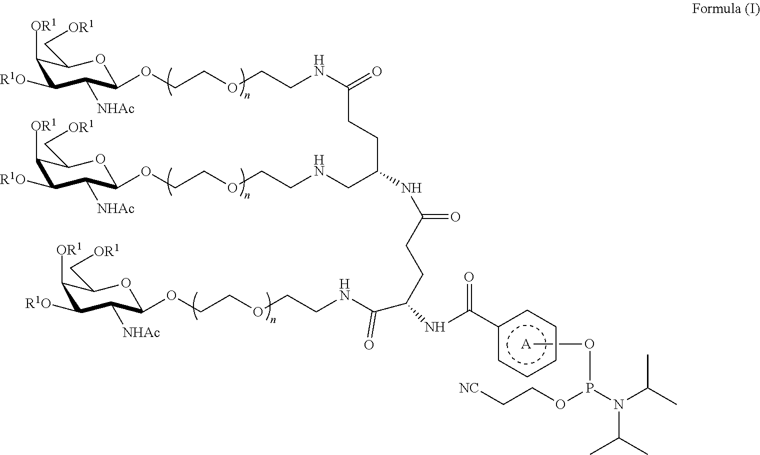

or a salt or stereoisomer thereof,
the process comprising Step (i): combining a compound of Formula (1):

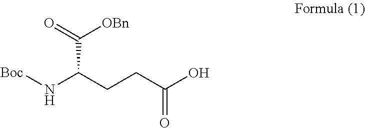 [SMILES: CC(C)(C)OC(=O)N[C@@H](CCC(=O)O)C(=O)OCc1ccccc1]

or a salt or stereoisomer thereof,
and a compound of Formula (2):

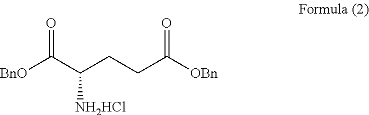

or a salt or stereoisomer thereof, to obtain a compound of Formula (3):

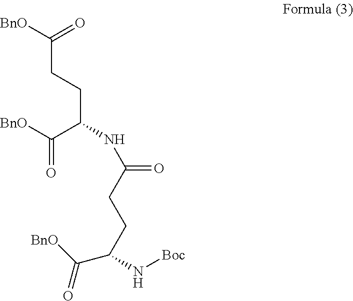 [SMILES: CC(C)(C)OC(=O)N[C@@H](CCC(=O)N[C@@H](CCC(=O)OCc1ccccc1)C(=O)OCc1ccccc1)C(=O)OCc1ccccc1]

or a salt or stereoisomer thereof. This reaction is illustrated in the following scheme:

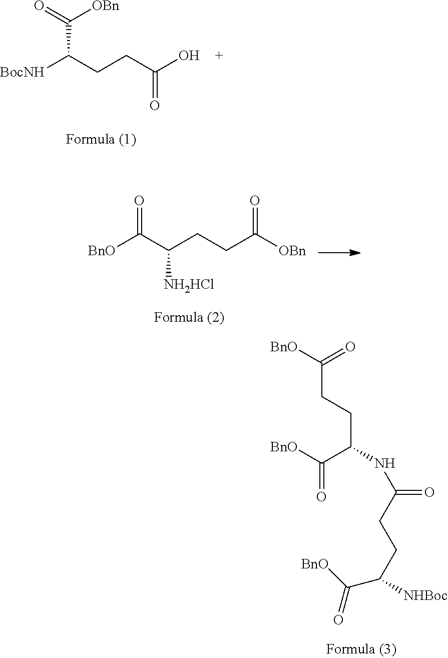

In some embodiments, Step (i) is performed in the presence of a base. In some embodiments, the base is pyridine, DMAP, triethylamine, isopropylethylamine, imidazole, DABCO, DBU, 2,6-lutidine, N-methylimidazole, NMM, or DIPEA. In some embodiments, the base is NMM.

In some embodiments, Step (i) is performed in the presence of an agent. In some embodiments, the agent is EDCI, isobutyl chloroformate, HOBt, HBTU, propylphosphonic anhydride, CDI, HOPO, or a mixture thereof. In some embodiments, the agent is isobutyl chloroformate. In some embodiments, the agent is CDI.

In some embodiments, Step (i) is performed in solution with some, or all of the reaction components dissolved and/or suspended in one or more solvents. In some embodiments, the solvent is a polar solvent. In some embodiments, the solvent is a polar, protic solvent. In some embodiments, the solvent is an alcohol. In some embodiments, the solvent is methanol, ethanol, isopropyl alcohol, isopropyl acetate, or butanol. In some embodiments, the solvent is isopropyl acetate. In other embodiments, the solvent is an aprotic solvent. In some embodiments, the solvent comprises acetonitrile, MethylTHF, Ethyl acetate, THF, or mixtures thereof.

In some embodiments, the process further comprises Step (ii): deprotecting three Bn groups from the compound of Formula (3), or a salt or stereoisomer thereof, to obtain a tri-acid compound of Formula (4):

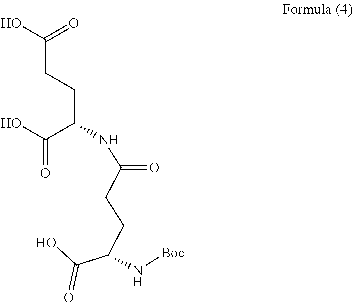 [SMILES: CC(C)(C)OC(=O)N[C@@H](CCC(=O)N[C@@H](CCC(=O)O)C(=O)O)C(=O)O]

or a salt or stereoisomer thereof. An embodiment of this reaction is illustrated in the following scheme:

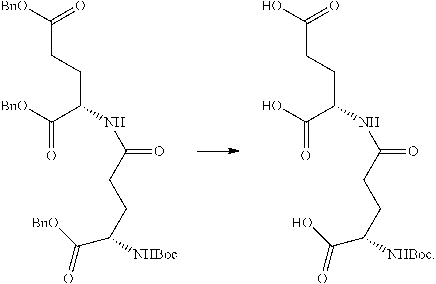 [SMILES: CC(C)(C)OC(=O)N[C@@H](CCC(=O)N[C@@H](CCC(=O)OCc1ccccc1)C(=O)OCc1ccccc1)C(=O)OCc1ccccc1 ; CC(C)(C)OC(=O)N[C@@H](CCC(=O)N[C@@H](CCC(=O)O)C(=O)O)C(=O)O]

In some embodiments, Step (ii) is performed under reductive conditions. In some embodiments, the step of deprotecting the Bn groups is performed under reductive conditions using a catalyst and hydrogen gas. In some embodiments, the catalyst is Pd/C. In some embodiments, the step of deprotecting the Bn groups is performed under hydrogen gas. In some embodiments, the pressure of hydrogen gas is about atmospheric pressure, about 10-15 psi, or about 40-50 psi.

In some embodiments, Step (ii) is performed in solution with some, or all of the reaction components dissolved and/or suspended in one or more solvents. In some embodiments, the solvent comprises acetonitrile, acetone, dichloromethane dimethylformamide, dimethylsulfoxide, N-methyl-2-pyrrolidone, 1,4-dioxane, or tetrahydrofuran, or mixtures thereof. In some embodiments, the solvent comprises tetrahydrofuran.

In some embodiments, a ti-acid compound of Formula (4), or a salt or stereoisomer thereof, in Step (ii) is purified by crystallization. In some embodiments, the crystallization is performed in a solvent, which comprises at least one selected from the group consisting of acetonitrile (MeCN), tetrahydrofuran (THF), isopropylacetate (IPAc), water, isopropyl alcohol (IPA), and any combination thereof. In some embodiments, the solvent is MeCN. In some embodiments, the solvent is MeCN in combination with other solvents, such as tetrahydrofuran (THF), isopropylacetate (IPAc), water, isopropyl alcohol (IPA).

In some embodiments, the process further comprises step (iii) coupling an N-acetyl-galactosamine derivative of Formula (X-a):

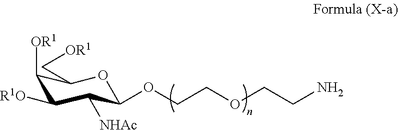

or a salt thereof (e.g., Formula (X-a) is in a salt form with a C₈-C₃₀ compound, such as the tri-acid of Formula (4),
to the tri-acid compound of Formula (4) or a salt or stereoisomer thereof, to form a tri-N-acetyl-galactosamines (tri-NAG) compound of Formula (5):

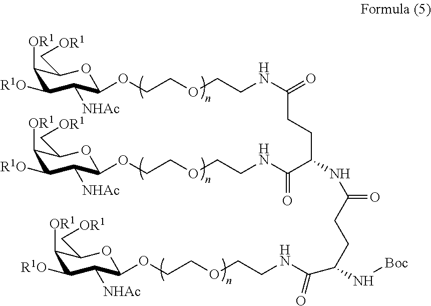

or a salt or stereoisomer thereof. An embodiments of this reaction is illustrated in the following scheme:

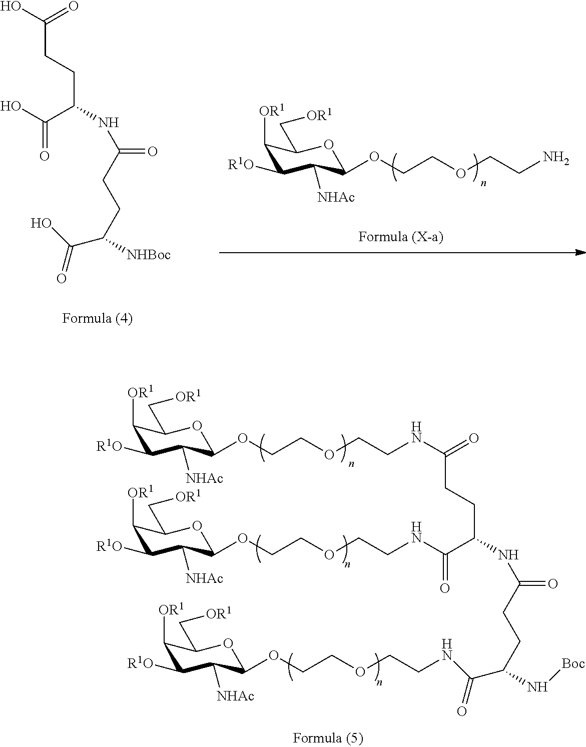

In some embodiments of step (iii), the salt of Formula (X-a) is an ammonium trifluoroacetate salt. In some embodiments, the compound of Formula (X-a) is in a salt form with a C₈-C₃₀ compound, such as the ti-acid of Formula (4).

In some embodiments, step (iii) is performed in the presence of a base. In some embodiments, the base is pyridine, DMAP, triethylamine, isopropylethylamine, imidazole, DABCO, DBU, 2,6-lutidine, N-methylimidazole, NMM, or DIPEA. In some embodiments, the base is DIPEA.

In some embodiments, Step (iii) is performed in the presence of an agent. In some embodiments, the agent is EDCI, isobutyl chloroformate, HOBt, HBTU, propylphosphonic anhydride, CDI, HOPO, or a mixture thereof. In some embodiments, the agent is isobutyl chloroformate. In some embodiments, the agent is hydroxybenzotriazole. In some embodiments, the agent is HBTU.

In some embodiments, step (iii) is performed in solution with some, or all of the reaction components dissolved and/or suspended in one or more solvents. In some embodiments, the solvent is acetonitrile, acetone, dichloromethane dimethylformamide, dimethylsulfoxide, N-methyl-2-pyrrolidone, 1,4-dioxane, or tetrahydrofuran. In some embodiments, the solvent is tetrahydrofuran. In some embodiments, the solvent is dichloromethane. In some embodiments, the solvent is dimethylformamide. In some embodiments, the solvent is a mixture of dichloromethane and dimethylformamide.

In some embodiments, the N-acetyl-galactosamine derivative of Formula (X-a), or a salt thereof, that is used in Step (iii) is obtained by deprotecting a benzyloxycarbonyl (Cbz) group of a protected N-acetyl-galactosamine derivative of Formula (X-f), in the presence of a C₈-C₃₀ compound, such as the tri-acid of Formula (4),

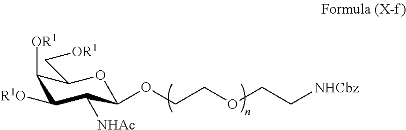

or a salt thereof. In some embodiments, the salt is a C₈-C₃₀ compound, such as the tri-acid of Formula (4),of the N-acetyl-galactosamine derivative of Formula (X-a).

In some embodiments, the salt is a trifluoroacetic acid (TFA) salt. In some embodiments, the step of deprotecting the Cbz group is conducted in flow chemistry. In some embodiments, the step of deprotecting the Cbz group is performed under reductive conditions. In some embodiments, the step of deprotecting the Cbz group is performed under reductive conditions using a catalyst and hydrogen gas. In some embodiments, the catalyst is Pd/C. In some embodiments, the step of deprotecting the Cbz group is performed under reductive conditions using a catalyst and hydrogen gas, and an acid additive. In some embodiments, the acid additive is toluenesulfonic acid, methanesulfonic acid, hydrochloric acid, sulfuric acid, trifluoroacetic acid, or a C₈-C₃₀ compound, such as the tri-acid of Formula (4). In some embodiments, the acid additive is trifluoroacetic acid.

In some embodiments, the step of deprotecting the Cbz group is performed under hydrogen gas. In some embodiments, the pressure of hydrogen gas is about atmospheric pressure, about 10-15 psi, or about 40-50 psi.

In some embodiments, the step of deprotecting the Cbz group is performed in solution with some, or all of the reaction components dissolved and/or suspended in one or more solvents. In some embodiments, the solvent is tetrahydrofuran, dichloromethane, or isopropanol. In some embodiments, the solvent is a mixture of tetrahydrofuran, dichloromethane, or isopropanol. In some embodiments, the solvent is tetrahydrofuran. In some embodiments, the solvent is isopropanol.

In some embodiments, Step (iii) and the step of deprotecting the Cbz group are performed together (e.g., without fully isolating a purified intermediate and/or deprotected in the presence of the C₈-C₃₀ compound). In some embodiments, Steps (ii) and (iii), and the step of deprotecting the Cbz group are performed together (e.g., without fully isolating a purified intermediate and/or deprotected in the presence of the C₈-C₃₀ compound).

In some embodiments, the process further comprises Step (iv) deprotecting a tert-butyloxycarbonyl (Boc) group of the tri-N-acetyl-galactosamine (tri-NAG) compound of Formula (5) or a salt or stereoisomer thereof, to obtain an tri-N-acetyl-galactosamine (tri-NAG) compound of Formula (6):

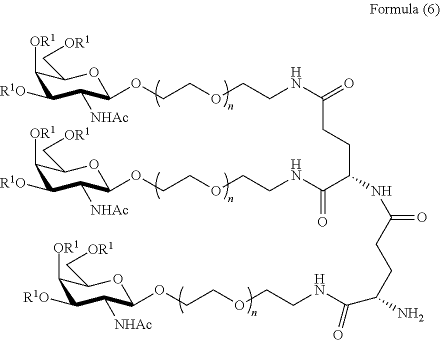

or a salt or stereoisomer thereof. An embodiment of this reaction is illustrated in the following scheme:

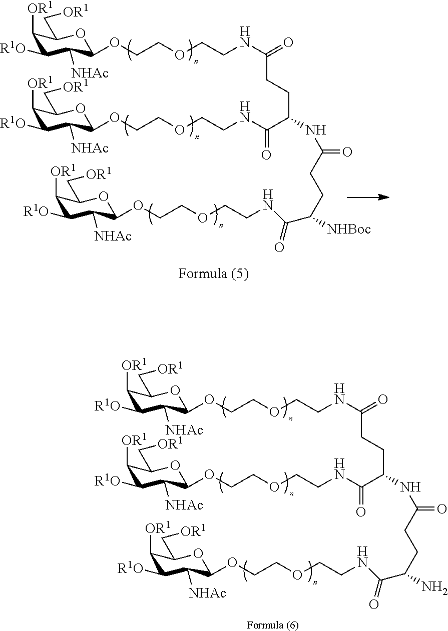

In some embodiments, Step (iv) is performed using a Lewis acid. In some embodiments, the Lewis acid is a trialkylsilyl trihalalkylsufonate. In some embodiments, the Lewis acid is trimethylsilyl trifluoromethanesulfonate (TMSOTf). In some embodiments, the Lewis acid also functions as a silylating agent. In some embodiments, Step 2 is performed with a dehydrating agent. In some embodiments, the dehydrating agent is an acetamide derivative. In some embodiments, the dehydrating agent is N,O-bis(trimethylsilyl)acetamide (BSA). In some embodiments, the dehydrating agent also functions as a silylating agent. In some embodiments, Step 2 is performed with both TMSOTf and BSA. In some embodiments, Step 2 is performed without BSA and/or with only TMSOTf. In some embodiments, Step 2 is performed using azeotropic distillation.

In some embodiments, Step (iv) is performed in solution with some, or all of the reaction components dissolved and/or suspended in one or more solvents. In some embodiments, the solvent is acetonitrile, acetone, dichloromethane dimethylformamide, dimethylsulfoxide, N-methyl-2-pyrrolidone, 1,4-dioxane, tetrahydrofuran, or a mixture thereof. In some embodiments, the solvent comprises acetonitrile. In some embodiments, the solvent comprises acetonitrile and at least one additional solvent (e.g., dichloromethane).

In some embodiments, the process further comprises Step (v) reacting the tri-N-acetyl-galactosamines (tri-NAG) compound of Formula (6), or a salt or stereoisomer thereof, with an acid of Formula (X-c):

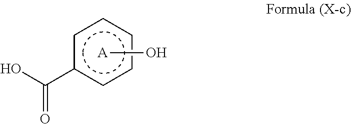

or a salt or stereoisomer thereof,
through a condensation reaction between the primary amine of the tri-N-acetyl-galactosamines (tri-NAG) compound of Formula (6) and the carboxylic acid of the acid of Formula (X-c) or a salt or stereoisomer thereof, to obtain a tri-N-acetyl-galactosamines (tri-NAG) compound of Formula (7),

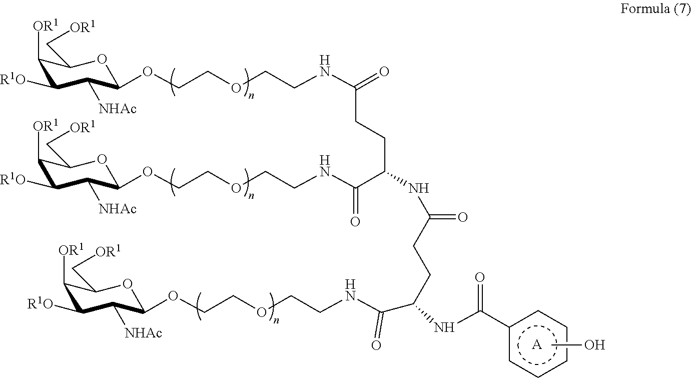

or a salt or stereoisomer thereof. This reaction is illustrated in the following scheme:

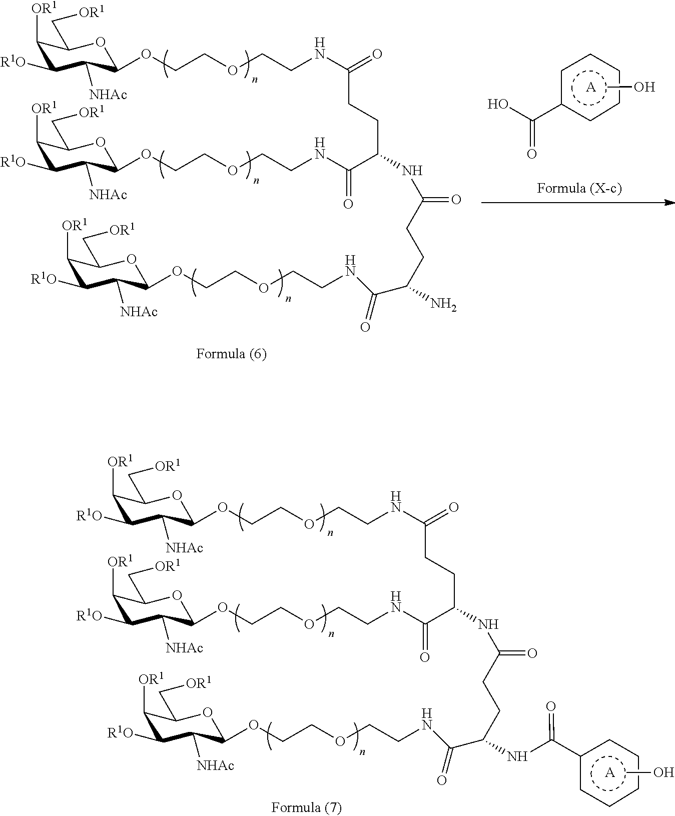

In some embodiments, Step (v) is performed in the presence of a base. In some embodiments, the base is pyridine, DMAP, triethylamine, isopropylethylamine, imidazole, DABCO, DBU, 2,6-lutidine, N-methylimidazole, NMM, or DIPEA. In some embodiments, the base is DIPEA.

In some embodiments, Step (v) is performed in the presence of an agent. In some embodiments, the agent is EDCI, isobutyl chloroformate, HOBt, HBTU, propylphosphonic anhydride, CDI, HOPO, or a mixture thereof. In some embodiments, the agent is EDCI. In some embodiments, the agent is hydroxybenzotriazole. In some embodiments, the agent is 2-hydroxypyridine-N-oxide. In some embodiments, the agents are EDCI and hydroxybenzotriazole. In some embodiments, the agents are EDCI and 2-hydroxypyridine-N-oxide.

In some embodiments, Step (v) is performed in solution with some, or all of the reaction components dissolved and/or suspended in one or more solvents. In some embodiments, the solvent is acetonitrile, acetone, dichloromethane, dimethylformamide, dimethylsulfoxide, N-methyl-2-pyrrolidone, 1,4-dioxane, tetrahydrofuran, or a mixture thereof. In some embodiments, the solvent is acetonitrile. In some embodiments, the solvent is dichloromethane.

In some embodiments, the process further comprises Step (vi) linking the tri-N-acetyl-galactosamines (tri-NAG) compound of Formula (7) or a salt or stereoisomer thereof, to a phosphorus atom of a phosphoramidite reagent through a phosphitylation reaction forming the phosphoramidite compound of Formula (I), or a salt or stereoisomer thereof, according to the following reaction scheme:

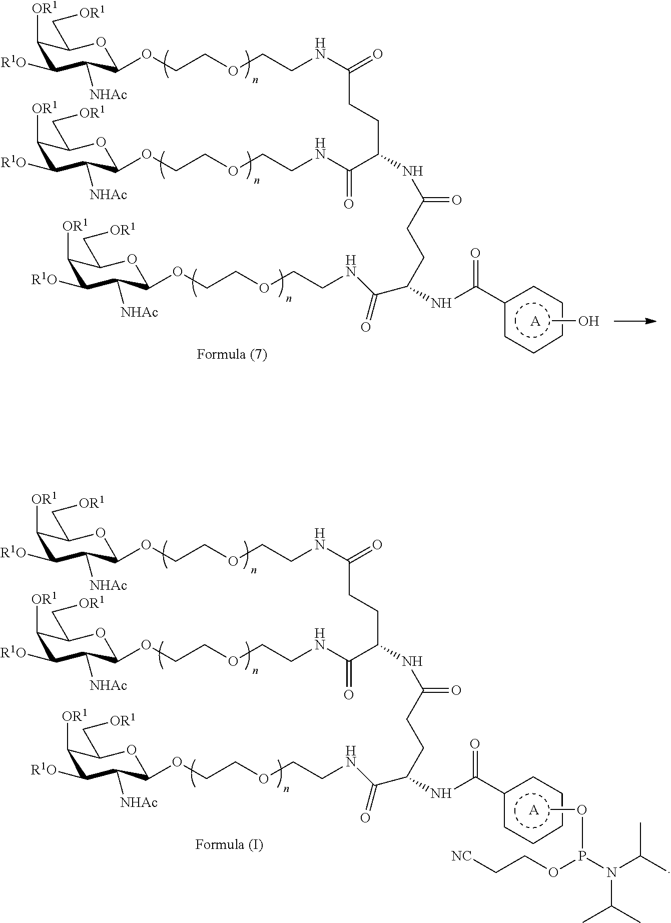

In some embodiments, Step (vi) is conducted in the presence of an agent. In some embodiments, the activating agent is tetrazole, ethylthiotetrazole (ETT), benzylthiotetrazole (BTT), N-methylimidazole (NMI) or dicyanoimidazole (DCI), and combination thereof. In some embodiments, the agent is tetrazole.

In some embodiments of Step (vi), the molar ratio of the phosphoramidite reagent to the tri-NAG compound of Formula (7) or a salt or stereoisomer thereof, is about 1.5.

In some embodiments, the phosphoramidite compound of Formula (I) or a salt or stereoisomer thereof is dried and stored below room temperature.

In some embodiments, Step (vi) is performed in solution with some, or all of the reaction components dissolved and/or suspended in one or more solvents. In some embodiments, the solvent is acetonitrile, acetone, dichloromethane dimethylformamide, dimethylsulfoxide, N-methyl-2-pyrrolidone, 1,4-dioxane, or tetrahydrofuran. In some embodiments, the solvent is dichloromethane.

In some embodiments, a method is provided for preparing a compound of Formula (I) that comprises performing Steps (ii) through (vi). In some embodiments, the method comprises performing Steps (i) through (v). In some embodiments, the method comprises performing Steps (i) through (iv). In some embodiments, the method comprises performing Steps (i) through (iii). In some embodiments, the method comprises performing Steps (i) and (ii). In some embodiments, the method comprises performing Steps (ii) through (vi). In some embodiments, the method comprises performing Steps (ii) through (v). In some embodiments, the method comprises performing Steps (ii) through (iv). In some embodiments, the method comprises performing Steps (ii) and (iii). In some embodiments, the method comprises performing Steps (iii) through (vi). In some embodiments, the method comprises performing Steps (iii) through (v). In some embodiments, the method comprises performing Steps (iii) and (iv). In some embodiments, the method comprises performing Steps (iv) through (vi). In some embodiments, the method comprises performing Steps (v) and (vi). In some embodiments, any two or more of Steps (i), (ii), (iii), (iv), (v), and (vi) are performed together (e.g., without fully isolating a purified intermediate and/or in the presence of a chemical reactant from another step).

In some embodiments, R¹ is acetyl. In some embodiments, n is 1. In some embodiments, R¹ is acetyl and n is 1.

In some embodiments, a method is provided for preparing a compound of Formula (X-1),

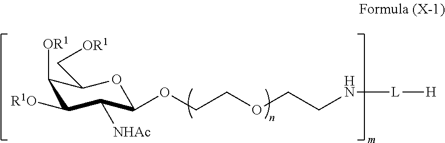

or a salt or stereoisomer thereof, wherein:
R¹ is H or acetyl;
n is an integer from 0 to 4;
m is an integer from 3 to 6;
p is an integer from 1 to 3;
Boc is tert-butyloxycarbonyl and
   L is a branched linker selected from the group consisting of:

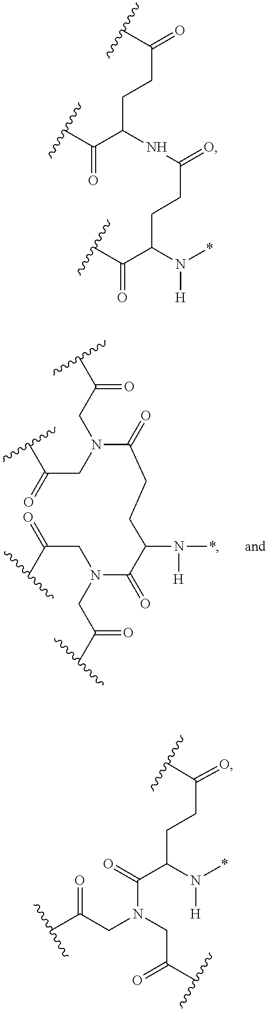

wherein the (*) indicates the attachment point to the hydrogen and the wavy line indicates an attachment point to the remaining of the poly-NAG compound, or a salt or stereoisomer thereof via nitrogen.

In some embodiments, R¹ is H or acetyl. In some embodiments, R¹ is H. R¹ is acetyl. In some embodiments, m is 3, 4, 5, or 6. In some embodiments, m is 3. In some embodiments, m is 4. In some embodiments, m is 5. In some embodiments, m is 6. In some embodiments, n is 1, 2, 3, or 4. In some embodiments, n is 1. In some embodiments, n is 2. In some embodiments, n is 3. In some embodiments, n is 4. In some embodiments, p is 1, 2, or 3. In some embodiments, p is 1. In some embodiments, p is 2. In some embodiments, p is 3.

In some embodiments, the method for preparing a compound of Formula (X-1) comprises Step (a): deprotecting benzyl (Bn) groups of a compound selected from the group consisting of

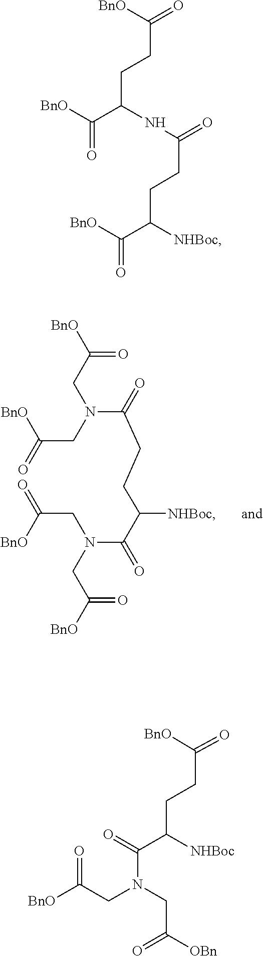 [SMILES: CC(C)(C)OC(=O)NC(CCC(=O)NC(CCC(=O)OCc1ccccc1)C(=O)OCc1ccccc1)C(=O)OCc1ccccc1 ; CC(C)(C)OC(=O)NC(CCC(=O)N(CC(=O)OCc1ccccc1)CC(=O)OCc1ccccc1)C(=O)N(CC(=O)OCc1ccccc1)CC(=O)OCc1ccccc1 ; CC(C)(C)OC(=O)NC(CCC(=O)OCc1ccccc1)C(=O)N(CC(=O)OCc1ccccc1)CC(=O)OCc1ccccc1]

to obtain a polyacid compound selected from the group consisting of

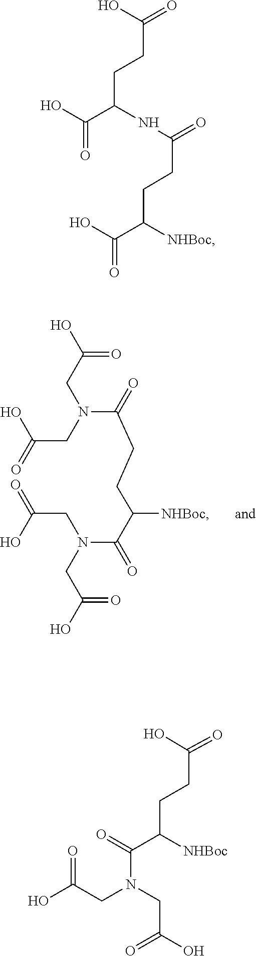 [SMILES: CC(C)(C)OC(=O)NC(CCC(=O)NC(CCC(=O)O)C(=O)O)C(=O)O ; CC(C)(C)OC(=O)NC(CCC(=O)N(CC(=O)O)CC(=O)O)C(=O)N(CC(=O)O)CC(=O)O ; CC(C)(C)OC(=O)NC(CCC(=O)O)C(=O)N(CC(=O)O)CC(=O)O]

In some embodiments, the method further comprises Step (b): coupling a N-acetyl-galactosamine derivative of Formula (X-a), or a salt thereof,

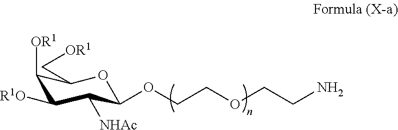

to the polyacid compound to form a poly-N-acetyl-galactosamines (poly-NAG) compound of Formula (X-2)

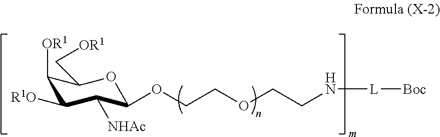

or a salt or stereoisomer thereof.

In some embodiments, the primary amine of the N-acetyl-galactosamine derivative of Formula (X-a) has been deprotected in the presence of the polyacid compound. In some embodiments, the N-acetyl-galactosamine derivative of Formula (X-a) and the polyacid compound form a salt.

In some embodiments, the method further comprises Step (c): deprotecting a tert-butyloxycarbonyl (Boc) group of the poly-N-acetyl-galactosamines (poly-NAG) compound of Formula (X-2) or a salt or stereoisomer thereof, to obtain the poly-N-acetyl-galactosamines (poly-NAG) compound of Formula (X-1) or a salt or stereoisomer thereof.

In some embodiments, the method further comprises Step (d): reacting the poly-N-acetyl-galactosamines (poly-NAG) compound of Formula (X-1) or a salt or stereoisomer thereof, with an acid of Formula (X-c)

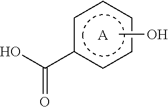

or a salt or stereoisomer thereof, wherein ring A is cyclohexanyl or phenyl, through a condensation reaction between the primary amine group of the compound of Formula (X-1) or a salt or stereoisomer thereof, and the carboxyl group of the acid of Formula (X-c) or a salt or stereoisomer thereof, to obtain a poly-N-acetyl-galactosamines (poly-NAG) compound of Formula (X-3)

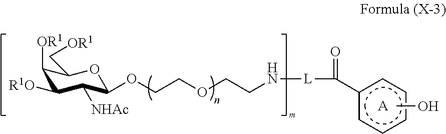

or a salt or stereoisomer thereof.

In some embodiments, ring A is cyclohexanyl. In some embodiments, ring A is phenyl.

In some embodiments, the method further comprises Step (e): linking the poly-N-acetyl-galactosamines (poly-NAG) compound of Formula (X-3) or a salt or stereoisomer thereof, to a phosphorus atom of a phosphoramidite reagent through a phosphitylation reaction forming the phosphoramidite compound of Formula (X-4)

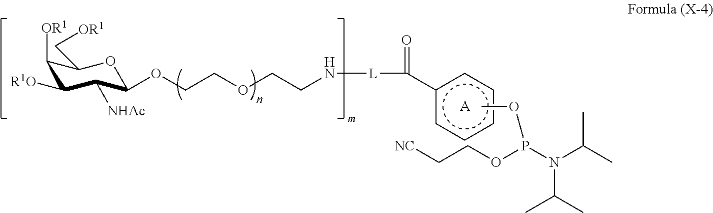

or a salt or stereoisomer thereof.

In some embodiments, the method further comprises Step (f): covalently attaching a therapeutic agent via the phosphorus atom to the phosphoramidite compound of Formula (X-4) or a salt or stereoisomer thereof.

In some embodiments, a method is provided for preparing a phosphoramidite compound of Formula (II)

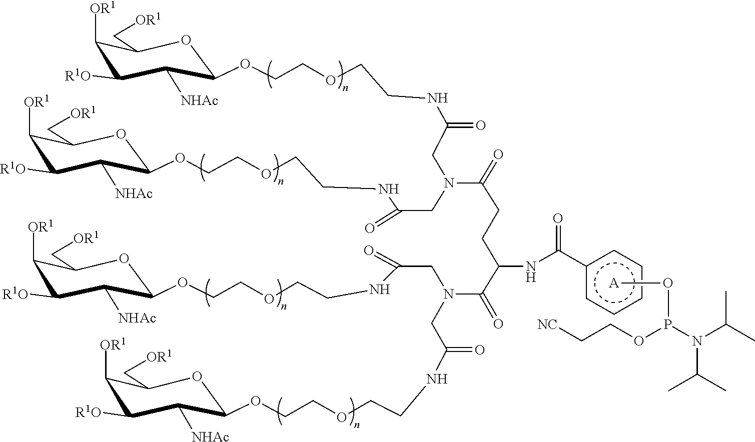

or a salt or stereoisomer thereof, wherein R¹ is H or acetyl, n is an integer from 0 to 4, and ring A is cyclohexanyl or phenyl. In some embodiments, R¹ is H or acetyl. In some embodiments, R¹ is H. R¹ is acetyl. In some embodiments, n is 1, 2, 3, or 4. In some embodiments, n is 1. In some embodiments, n is 2. In some embodiments, n is 3. In some embodiments, n is 4. In some embodiments, ring A is cyclohexanyl or phenyl. In some embodiments, ring A is cyclohexanyl. In some embodiments, ring A is phenyl.

In some embodiments, the primary amine of the N-acetyl-galactosamine derivative of Formula (X-a) has been deprotected in the presence of the quad-acid compound. In some embodiments, the N-acetyl-galactosamine derivative of Formula (X-a) and the quad-acid compound form a salt.

In some embodiments, the method comprises step (A): reacting

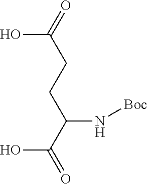 [SMILES: CC(C)(C)OC(=O)NC(CCC(=O)O)C(=O)O]

with

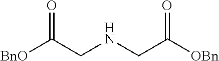 [SMILES: O=C(CNCC(=O)OCc1ccccc1)OCc1ccccc1]

to obtain a compound having a structure of

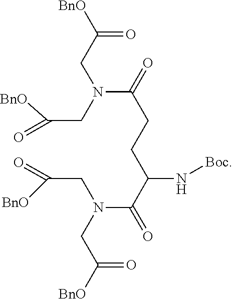 [SMILES: CC(C)(C)OC(=O)NC(CCC(=O)N(CC(=O)OCc1ccccc1)CC(=O)OCc1ccccc1)C(=O)N(CC(=O)OCc1ccccc1)CC(=O)OCc1ccccc1]

In some embodiments, the method further comprises Step (B): deprotecting three benzyl (Bn) groups of the compound from the step (i) to obtain a quad-acid compound having a structure of,

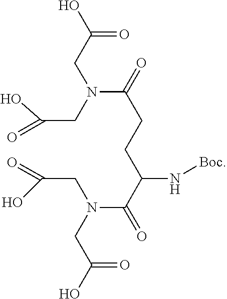 [SMILES: CC(C)(C)OC(=O)NC(CCC(=O)N(CC(=O)O)CC(=O)O)C(=O)N(CC(=O)O)CC(=O)O]

In some embodiments, the method further comprises Step (C): coupling a N-acetyl-galactosamine derivative of Formula (X-a),

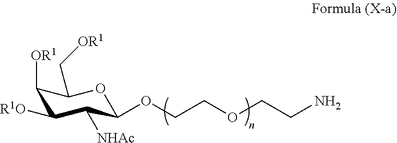

or a salt thereof, to the quad-acid compound from the step (B) to form a compound having a structure of

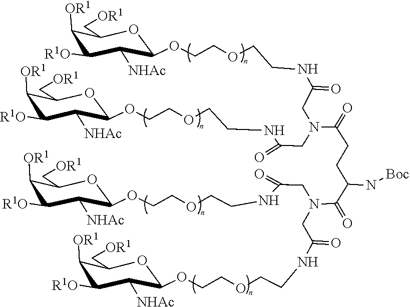

In some embodiments, the method further comprises Step (D): deprotecting a tert-butyloxycarbonyl (Boc) group of the compound from the step (iii) to obtain a compound having a structure of

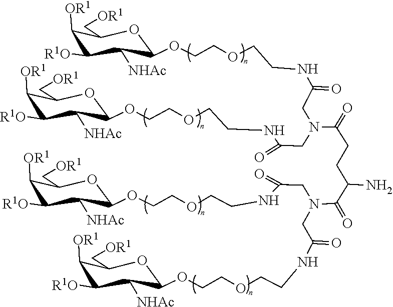

In some embodiments, the method further comprises Step (E): reacting the compound from the step (iv) with an acid of Formula (X-c)

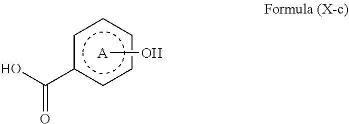

or a stereoisomer thereof, through a condensation reaction between the primary amine group of the compound from the step (D) and the carboxyl group of the acid of Formula (X-c) or a stereoisomer thereof, to obtain a quad-N-acetyl-galactosamines (quad-NAG) compound having a structure of

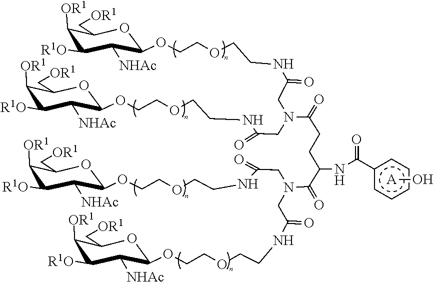

In some embodiments, the method further comprises Step (F): linking the quad-N-acetyl-galactosamines (quad-NAG) compound from the step (E) to a phosphorus atom of a phosphoramidite reagent through a phosphitylation reaction forming the phosphoramidite compound of Formula (II) or a salt or stereoisomer thereof.

In some embodiments, provided is a method for preparing a phosphoramidite compound of Formula (III)

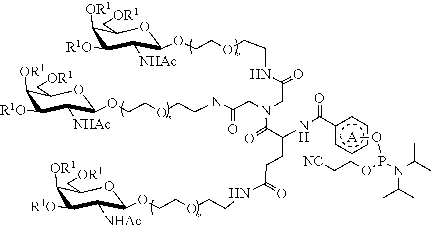

or a salt or stereoisomer thereof, wherein R¹ is H or acetyl, n is an integer from 0 to 4, and ring A is cyclohexanyl or phenyl. In some embodiments, R¹ is H or acetyl. In some embodiments, R¹ is H. R¹ is acetyl. In some embodiments, n is 1, 2, 3, or 4. In some embodiments, n is 1. In some embodiments, n is 2. In some embodiments, n is 3. In some embodiments, n is 4. In some embodiments, ring A is cyclohexanyl or phenyl. In some embodiments, ring A is cyclohexanyl. In some embodiments, ring A is phenyl.

In some embodiments, the method comprises Step (I): reacting

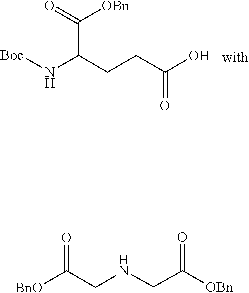 [SMILES: CC(C)(C)OC(=O)NC(CCC(=O)O)C(=O)OCc1ccccc1 ; O=C(CNCC(=O)OCc1ccccc1)OCc1ccccc1]

to obtain a compound having a structure of

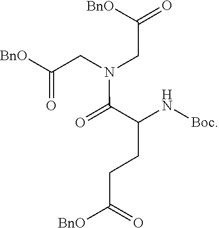 [SMILES: CC(C)(C)OC(=O)NC(CCC(=O)OCc1ccccc1)C(=O)N(CC(=O)OCc1ccccc1)CC(=O)OCc1ccccc1]

In some embodiments, the method further comprises Step (II): deprotecting three benzyl (Bn) groups of the compound from the Step (I) to obtain a tri-acid compound having a structure of

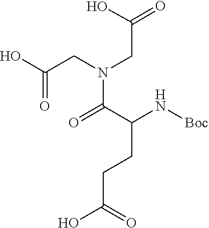 [SMILES: CC(C)(C)OC(=O)NC(CCC(=O)O)C(=O)N(CC(=O)O)CC(=O)O]

In some embodiments, the method further comprises Step (III): coupling a N-acetyl-galactosamine derivative of Formula (X-a)

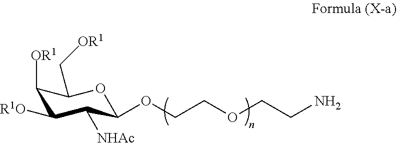

or a salt thereof, to the ti-acid compound from the Step (II) to form a compound having a structure of

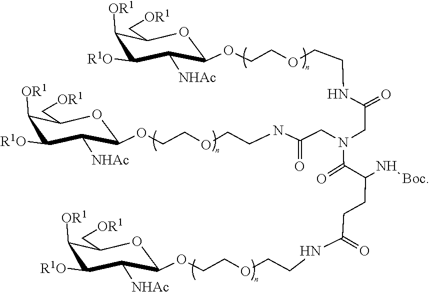

In some embodiments, the primary amine of the N-acetyl-galactosamine derivative of Formula (X-a) has been deprotected in the presence of the tri-acid compound. In some embodiments, the N-acetyl-galactosamine derivative of Formula (X-a) and the tri-acid compound form a salt.

In some embodiments, the method further comprises Step (IV): deprotecting a tert-butyloxycarbonyl (Boc) group of the compound from the step (iii) to obtain a compound having a structure of

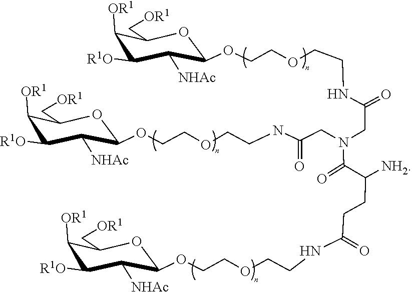

In some embodiments, the method further comprises Step (V): reacting the compound from the Step (IV) with an acid of Formula (X-c) or a stereoisomer thereof,

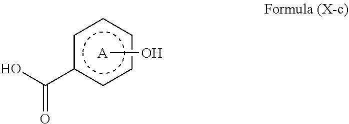

or a stereoisomer thereof, through a condensation reaction between the primary amine group of the compound from the Step (IV) and the carboxyl group of the acid of Formula (X-c) or a stereoisomer thereof, to obtain a tri-N-acetyl-galactosamines (tri-NAG) compound having a structure of

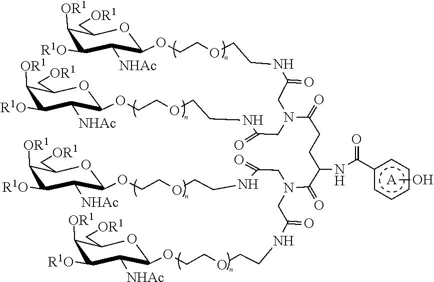

In some embodiments, the method further comprises Step (VI): linking the tri-N-acetyl-galactosamines (tri-NAG) compound from the step (v) to a phosphorus atom of a phosphoramidite reagent through a phosphitylation reaction forming the phosphoramidite compound of Formula (III) or a salt or stereoisomer thereof.

In another aspect, provided herein is a phosphoramidite compound of Formula (I)

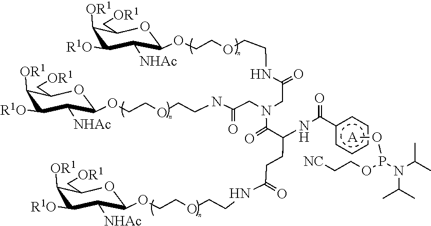

or a salt or stereoisomer thereof, wherein R¹ is H or acetyl, n is an integer from 0 to 4, and ring A is cyclohexanyl or phenyl. In some embodiments, R¹ is H. In some embodiments, R¹ is acetyl. In some embodiments, n is 0. In some embodiments, n is 1. In some embodiments, n is 2. In some embodiments, n is 3. In some embodiments, n is 4. In some embodiments, ring A is cyclohexanyl. In some embodiments, ring A is phenyl. In some embodiments, R¹ is acetyl, n is 1, and ring A is cyclohexanyl.

Also provided is a tri-N-acetyl-galactosamines (tri-NAG) compound having a structure of

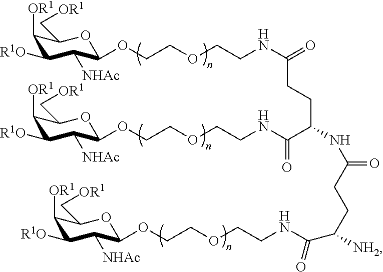

or a salt or stereoisomer thereof, wherein R¹ is H or acetyl, and n is an integer from 0 to 4. In some embodiments, R¹ is H. In some embodiments, R¹ is acetyl. In some embodiments, n is 0. In some embodiments, n is 1. In some embodiments, n is 2. In some embodiments, n is 3. In some embodiments, n is 4. In some embodiments, R¹ is acetyl and n is 1.

Further provided is a tert-butyloxycarbonyl (Boc) group protected tri-N-acetyl-galactosamines (tri-NAG) compound having a structure of

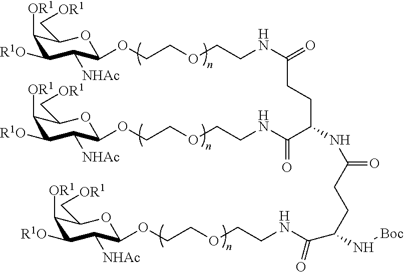

or a salt or stereoisomer thereof, wherein R¹ is H or acetyl, and n is an integer from 0 to 4. In some embodiments, R¹ is H. In some embodiments, R¹ is acetyl. In some embodiments, n is 0. In some embodiments, n is 1. In some embodiments, n is 2. In some embodiments, n is 3. In some embodiments, n is 4. In some embodiments, R¹ is acetyl and n is 1.

In some embodiments, an intermediate having the structure of

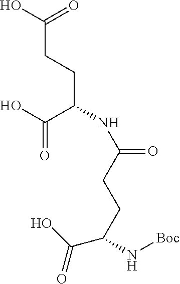 [SMILES: CC(C)(C)OC(=O)N[C@@H](CCC(=O)N[C@@H](CCC(=O)O)C(=O)O)C(=O)O]

or a salt or stereoisomer thereof is described. In some embodiments, the intermediate is in a crystalline form.

In some embodiments, an intermediate having the structure of

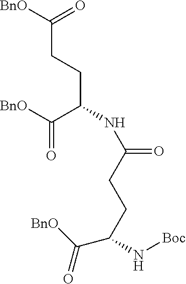 [SMILES: CC(C)(C)OC(=O)N[C@@H](CCC(=O)N[C@@H](CCC(=O)OCc1ccccc1)C(=O)OCc1ccccc1)C(=O)OCc1ccccc1]

or a salt or stereoisomer thereof, is described. In some embodiments, the intermediate is in a crystalline form.

In yet another aspect, the invention comprises a targeting ligand of Formula (X-e),

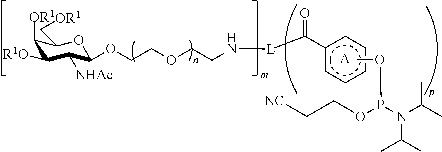

or a salt or stereoisomer thereof, which is prepared by the methods described herein.

In some embodiments, the targeting ligand of Formula (X-e) has a structure of

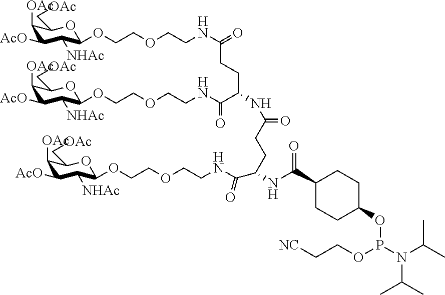

In some embodiments, the targeting ligands disclosed herein are linked to therapeutic compounds. In some embodiments, the targeting ligand is linked to the therapeutic compound via an additional linker and/or a cleavable moiety, which is then linked to the therapeutic compound. In some embodiments, targeting ligands are ligated to the therapeutic compound itself.

In some embodiments, the therapeutic compound is an oligomeric compound. In some embodiments, the therapeutic compound is an expression-inhibiting oligomeric compound. In some embodiments, the expression-inhibiting oligomeric compound is an RNAi agent. In some embodiments, the expression-inhibiting oligomeric compound is a double-stranded RNAi agent.

In some embodiments, a targeting ligand is linked directly or indirectly to the 5′ end of the sense strand of a double-stranded RNAi agent. In some embodiments, the targeting ligand is linked directly or indirectly to the 3′ end of the sense strand of a double-stranded RNAi agent. In some embodiments, the targeting ligand is linked directly or indirectly to the 5′ end or the 3′ end of the antisense strand of a double-stranded RNAi agent. In some embodiments, the targeting ligand-containing phosphoramidite is linked to the 5′ end of the sense strand of a double-stranded RNAi agent.

### EXAMPLES

The presently disclosed subject matter will be better understood by reference to the following Examples, which are provided as exemplary of the invention, and not by way of limitation.

### Abbreviations

ACN acetonitrile
Boc tert-butyloxycarbonyl
Bn benzyl
BSA N,O-bis(trimethylsilyl)acetamide
BTT benzylthiotetrazole
Cbz carboxybenzyl
CDI 1,1′-carbonyldiimidazole
DCI dicyanoimidazole
DABCO 1,4-diazabicyclo[2.2. 2]octane
DBU 1,8-Diazabicyclo[5.4.0]undec-7-ene
DIPEA N,N-diisopropylethylamine
DMAP 4,-dimethylaminopyridine
EDCI 1-ethyl-3-(3-dimethylaminopropyl)carbodiimide
ETT ethylthiotetrazole
HBTU 2-(1H-benzotriazol-1-yl)-1,1,3,3-tetramethyluronium hexauorophosphate
HOBt hydroxybenzotriazole
HOPO 2-hydroxypyridine-N-oxide
IPA isopropyl alcohol
IPAc isopropyl acetate
NAG N-acetyl-galactosamine
NMI N-methylimidazole
NMM N-methylmorpholine
MeCN acetonitrile
psi pounds per square inch
THF tetrahydrofuran
TMSOTf trimethylsilyl trifluoromethanesulfonate
RNAi RNA interference

### Example 1. Preparation of Formula (3)

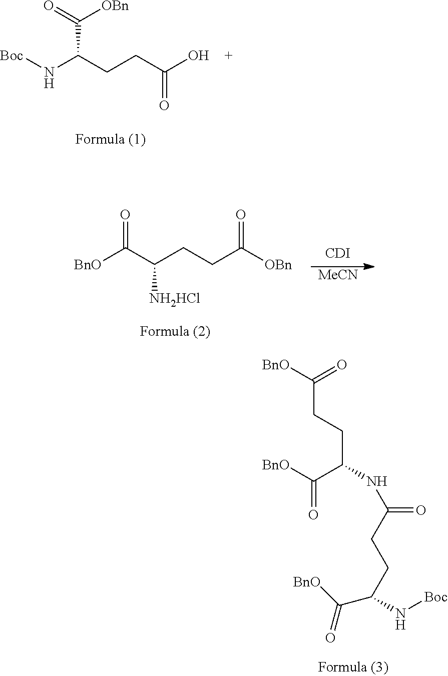

A mixture of Formula (1) (20 gram, 1.0 equiv.) in MeCN (120 mL, 6V) was stirred at 0-5° C. and CDI (10.52 gram, 1.1 equiv.) was added. The resulting solution was stirred at 0-5° C. and analyze by HPLC. Formula (2) (23.7 g, 1.1 eq.) was added batchwise into the reactor at 0-5° C. to afford a suspension. The suspension was warmed to 25-30° C. over 2 hours and stirred for about 16 hours and analyzed by HPLC. The reaction was quenched by addition of 5% citric acid (120 mL, 6V) dropwise at 25-30° C. to afford a suspension (observed solid precipitate during quench, pH=5-6). The mixture was cooled to 0-5° C. and stirred for 3 h at 0-5° C. The slurry was filtered, and the cake washed with water (20 mL*2, 1 V*2) and dried under vacuum at 45-50° C. for 3 h to afford 37 gram (93% yield) of Formula (3) as a white solid (purity: 95.7 A %, 97.1 wt. %).

¹H NMR (400 MHz, DMSO-d₆) δ ppm 1.14-1.32 (m, 2H) 1.37 (s, 7H) 1.72-1.91 (m, 2H) 1.94-2.07 (m, 2H) 2.23 (br t, J=7.46 Hz, 2H) 2.33-2.48 (m, 2H) 3.96-4.09 (m, 1H) 4.26-4.40 (m, 1H) 5.04-5.17 (m, 6H) 7.29-7.41 (m, 16H) 8.28 (br d, J=7.58 Hz, 1H)

### Example 2. Preparation of Formula (4)

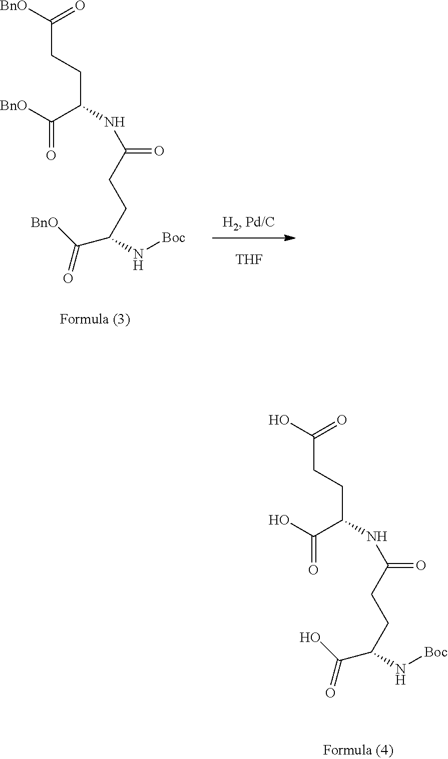 [SMILES: CC(C)(C)OC(=O)N[C@@H](CCC(=O)N[C@@H](CCC(=O)OCc1ccccc1)C(=O)OCc1ccccc1)C(=O)OCc1ccccc1 ; CC(C)(C)OC(=O)N[C@@H](CCC(=O)N[C@@H](CCC(=O)O)C(=O)O)C(=O)O ; "THF"]

A 5 L pressure reactor was charged with THF (1 L, 10 V, KF=0.02%) and tri-ester Formula (3) (100.0 g, 1.0 eq.) at 20-30° C. The reactor was purged with nitrogen and Pd/C (50% wet, 10 wt. % loading, 10 g, ca. 3 mol %) was added. The reactor was sealed and purged with hydrogen 3 times and pressurized with 40-50 psi hydrogen and stirred at 20-30° C. for 15 h. A sample was taken for analysis. The reaction mixture was filtered through diatomite and the cake was washed with THF (500 mL*2, 5V*2). The solution was concentrated at <40° C. to about 1-2V and stirred at 20-30° C. Isopropyl acetate (900 mL, 9V) was added over about 30 min. The resulting suspension was cooled to −10-0° C. and stirred for about 12 hours and filtered. The cake was washed with isopropyl acetate (300 mL*2, 3V*2) and dried under vacuum at 30-40° C. for 10-15 h to afford Formula (4) as a white solid. (Purity: 98%, Isolated yield: 93%).

¹H NMR (400 MHz, DMSO-d₆) δ ppm 1.35-1.38 (m, 9H) 1.67-1.80 (m, 2H) 1.84-1.99 (m, 2H) 2.17-2.34 (m, 4H) 3.58-3.87 (m, 1H) 4.16-4.22 (m, 1H) 6.71-7.07 (m, 1H) 8.07 (br d, J=7.82 Hz, 1H) 12.40 (br s, 3H).

### Example 3. Telescoped Procedure for the Preparation of Formula (4)

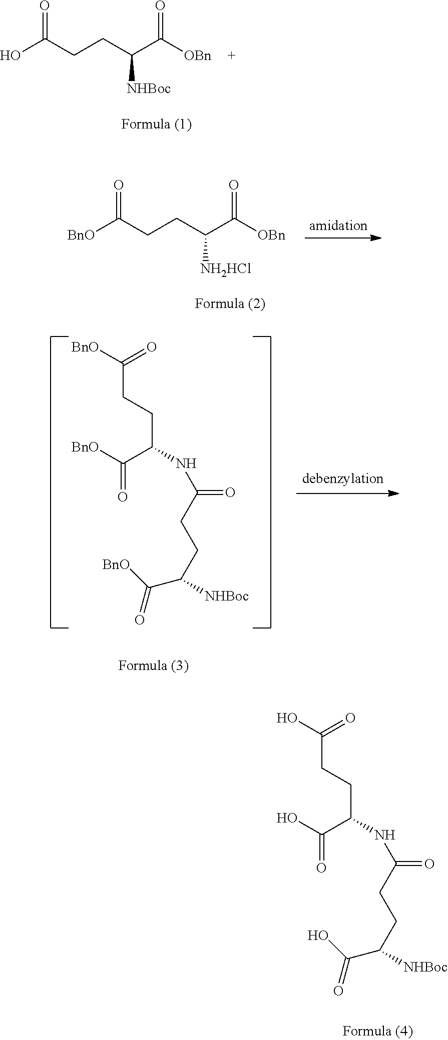

To a 5 L reactor under nitrogen was added Formula (1) (250.0 g, 1.0 eq.) and isopropyl acetate (2.5 L, 10 V, KF=0.03%). The suspension was cooled to 0-5° C. and CDI (126.2 g, 1.05 eq.) was charged into the reactor at 0-5° C. to afford a solution that was stirred at 0-5° C. for 1 h and analyzed by quenching a sample into MeOH. Formula (2) (296.6 g, 1.1 eq.) was added batchwise into reactor R1 at 0-5° C., leading to a suspension that was warmed to 25-30° C. over 2 hours and stirred for about 24 h. The reaction was quenched by slow addition of 10 wt. % aq. citric acid (1250 mL, 5 V) into R1 at 25-30° C. and stirred for 30 min. The layers were separated, and the organic layer washed with 10 wt. % aq. citric acid (1250 mL, 5 V), followed by water (1250 mL, 5 V). The organic layer was concentrated under vacuum to about 5 V and diluted with THF (2.5 L, 10 V) at 20-30° C. The solution was added to a pressure reactor and 10% wet Pd/C (12.5 g, 10 wt. %) was added at 20-30° C. The reactor was purged with hydrogen three times and the contents stirred under 40-50 psi hydrogen for about 20 hours. The reaction was analyzed, and the reaction mixture filtered through a pad of Celite (20 wt. %, 50 g). The cake was washed with THF (500 mL*3, 2 V*3) and the solution concentrated by vacuum distillation at 40-45° C. batch temperature to about 7 V. A solvent switch to acetonitrile was done by three put and take distillations with 4V acetonitrile to afford a slurry in about 4V acetonitrile that was stirred at 20-30° C. for 1 h and filtered. The cake was washed with acetonitrile (250 mL*2, 1V*2) and dried under vacuum at 45-50° C. for 2 h to obtain 248 gram (87.5% yield over 2-steps) of the triacid of Formula (4) as a white solid.

¹H NMR (400 MHz, DMSO-d₆) δ ppm 1.35-1.38 (m, 9H) 1.67-1.80 (m, 2H) 1.84-1.99 (m, 2H) 2.17-2.34 (m, 4H) 3.58-3.87 (m, 1H) 4.16-4.22 (m, 1H) 6.71-7.07 (m, 1H) 8.07 (br d, J=7.82 Hz, 1H) 12.40 (br s, 3H).

### Example 4. Procedure for the Preparation of Formula (10)

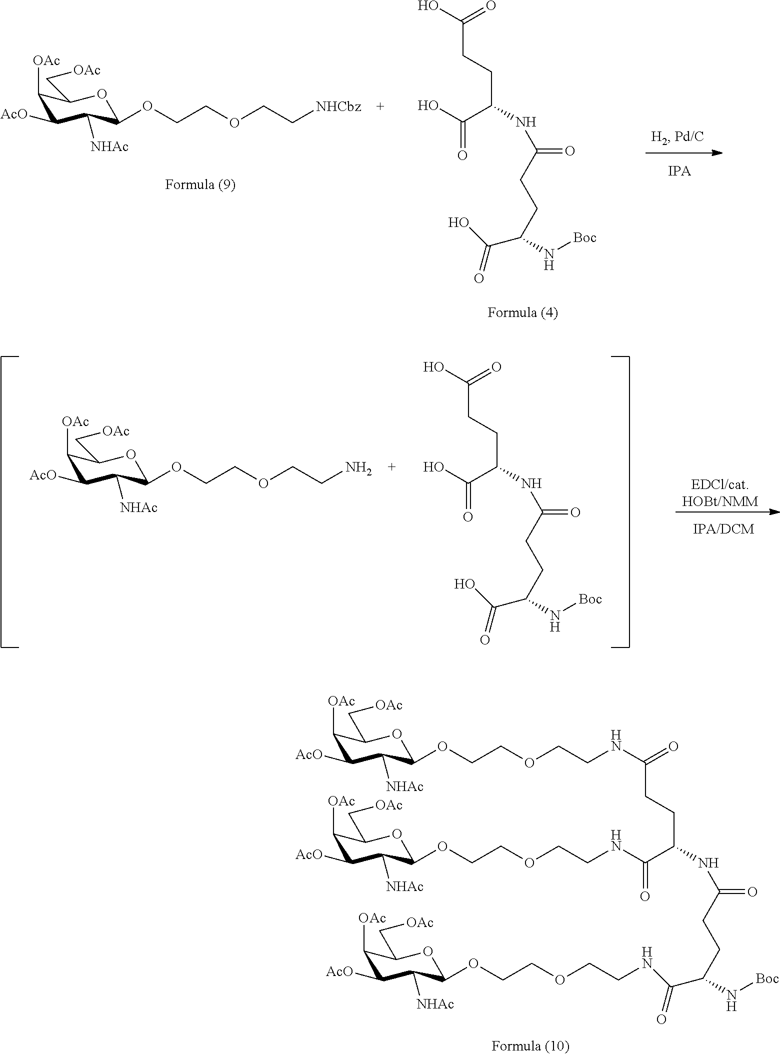

To a pressure reactor was added Formula (9) (200 g, 1.0 equiv.), Formula (4) (45.3 g, 0.33 equiv.) and isopropanol (5 vol, 1000 mL). The wet catalyst Pd/C (30 gram, 15% loading, 2.1 mol %) was charged and the sealed reactor swapped with 30-35 psi nitrogen three times followed by 30-35 psi hydrogen three times. The reactor was then pressurized with 30-35 psi and the contents stirred for 6 hours total at 20-30° C. with an intermediate sample after 2 hours stirring time. The reaction mixture was filtered, and the cake washed with dichloromethane (3×400 mL). The combined isopropanol/dichloromethane organic layer was charged to a reactor under nitrogen. The temperature was adjusted to 0-10° C. and N-methylmorpholine (160 gram, 4.5 equiv), HOBt (9.5 gram, 0.2 equiv.) and EDCI (269 gram, 4.0 equiv.) were charged into the reactor in the order given at 0-10° C. The reactor wall was rinsed with dichloromethane (450 mL). The batch temperature was adjusted to 20-30° C. and the mixture stirred for about 16 hours and analyzed for completion. Dichloromethane (1100 mL) was charged to the reactor followed by aq. 1M KH₂PO4 (870 mL). The layers were separated, and the organic layer was washed with aq. 1M K₂HPO4 (780 mL) two times followed by a wash with water (900 mL). The organic layer was concentrated under vacuum to about 900 mL, keeping batch temperature <45° C. A solvent switch was done by put and take vacuum distillation with 3×450 mL isopropanol, keeping batch temperature at <45° C. and reactor volume at about 900 mL. Methyl tert-butyl ether (2300 mL) was charged to the reactor at 40-50° C. over about 3 hours. The mixture was cooled to 20-25° C. over 2 hours and stirred for about 16 hours and filtered. The cake was washed with methyl tert-butyl ether (2×225 mL) and dried under vacuum at 50-60° C. for 20-24 hours to afford 175 gram of Formula (10) in 80-85% yield.

¹H NMR (400 MHz, DMSO-d₆) δ ppm 1.38 (s, 9H) 1.59-2.22 (m, 10H) 1.78-1.79 (m, 9H) 1.90 (s, 9H) 2.00 (s, 9H) 2.11 (s, 9H) 3.14-3.26 (m, 6H) 3.35-3.65 (m, 13H) 3.76-3.92 (m, 7H) 4.04 (s, 9H) 4.18 (br d, J=5.87 Hz, 1H) 4.56 (dd, J=8.44, 2.81 Hz, 3H) 4.97-5.02 (m, 3H) 5.22 (d, J=3.42 Hz, 3H) 6.84 (br d, J=8.07 Hz, 1H) 7.82 (br d, J=8.80 Hz, 5H) 7.90-7.96 (m, 2H)

### Example 5. Telescoped Procedure for the Preparation of Formula (10)

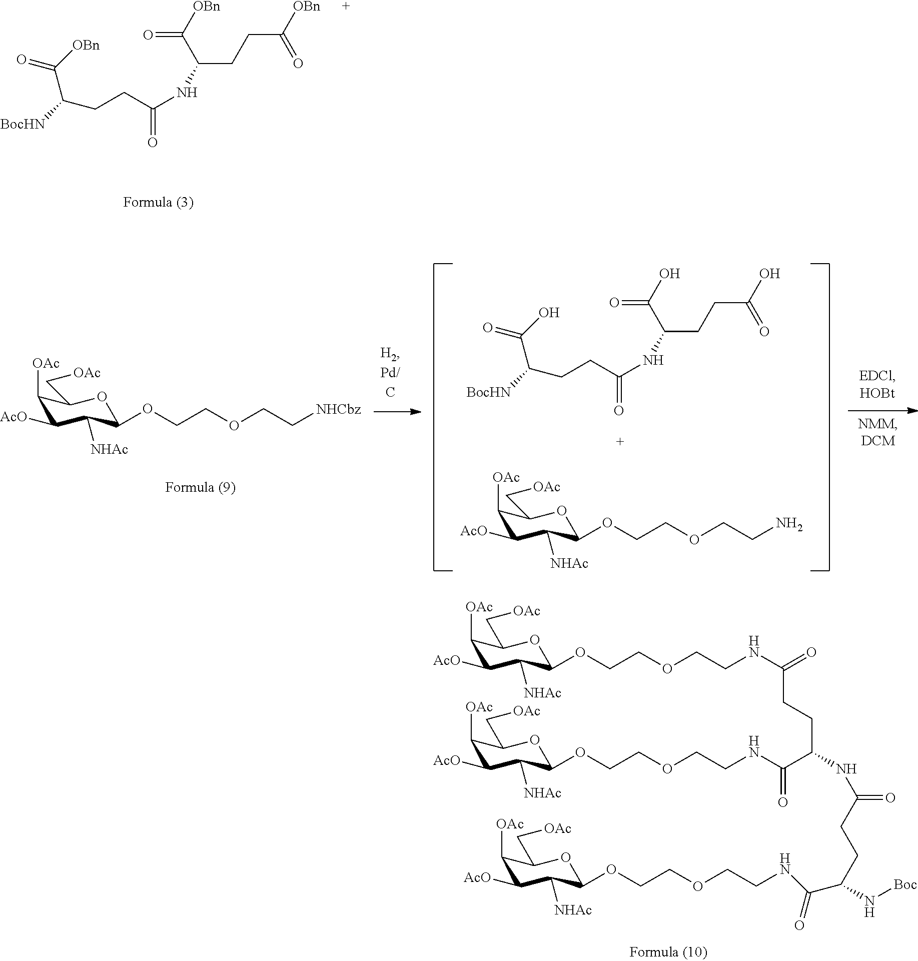

To a 1000 mL pressure reactor R1 under N₂ was added Formula (3) (17.1 g, 0.3 eq.), Formula (9) (50.0 g, 1.0 eq.) and THF (500 mL, 10 V). Palladium catalyst, 10% wet Pd/C (5 g, 10 wt. %) was charged into R1 at 20-30° C. and the reactor was purged with hydrogen 3 times. The mixture was stirred at 20-30° C. for about 24 h under 15-20 psi of H₂. The batch was filtered through a pad of Celite, and the cake washed with THF (100 mL*3, 2 V*3) and the filtrate concentrated under reduced pressure to about 1-2V at 20-30° C. DCM (500 mL, 10 V) was charged.

All of the following steps, amounts and volumes were against the triacid, assuming a 100% yield for the de-benzylation.

The mixture was cooled to 5-10° C. and NMM (12.1 g, 4.5 eq.) was added dropwise into reactor R1 at 5-10° C. HOBt (720 mg, 0.2 eq.) was charged into reactor R1 at 5-10° C. followed by EDCI (20.4 g, 4.0 eq.) at 5-10° C. The reaction was warmed to 20-25° C. and stirred for 2 h at 20-25° C. and analyzed for completion by HPLC. Aqueous 1M KH₂PO4 aq. (50 mL, 5 V) was added and the mixture stirred, and the layers separated. The organic phase was washed with 1M K₂HPO4 aq. twice (100 mL, 10 V*2) and once with water (50 mL, 5 V). To the organic phase was added IPA (80 mL, 8 V) and the mixture concentrated under reduced pressure to about 20 V at 40-45° C. The solvent switch to IPA was repeated three times (100 mL*3, 10 V*3) to afford about 20 V solution that was stirred at 25-30° C. MTBE (500 mL, 50 V) was charged slowly into the organic phase at 25-30° C. and the resulting suspension was stirred for 15 h at 25-30° C. and filtered. The cake was washed with MTBE (25 mL*2, 5 V*2). The wet cake was dried under vacuum at 50-55° C. for at least 4 h to obtain 29.5 gram of Formula (10) as a white solid (97 wt. % by Q-NMR, purity: 94.4 A %).

¹H NMR (400 MHz, DMSO-d₆) δ ppm 1.38 (s, 9H) 1.59-2.22 (m, 10H) 1.78-1.79 (m, 9H) 1.90 (s, 9H) 2.00 (s, 9H) 2.11 (s, 9H) 3.14-3.26 (m, 6H) 3.35-3.65 (m, 13H) 3.76-3.92 (m, 7H) 4.04 (s, 9H) 4.18 (br d, J=5.87 Hz, 1H) 4.56 (dd, J=8.44, 2.81 Hz, 3H) 4.97-5.02 (m, 3H) 5.22 (d, J=3.42 Hz, 3H) 6.84 (br d, J=8.07 Hz, 1H) 7.82 (br d, J=8.80 Hz, 5H) 7.90-7.96 (m, 2H)

### Example 6. Telescoped Procedure for the Preparation of Formula (12)

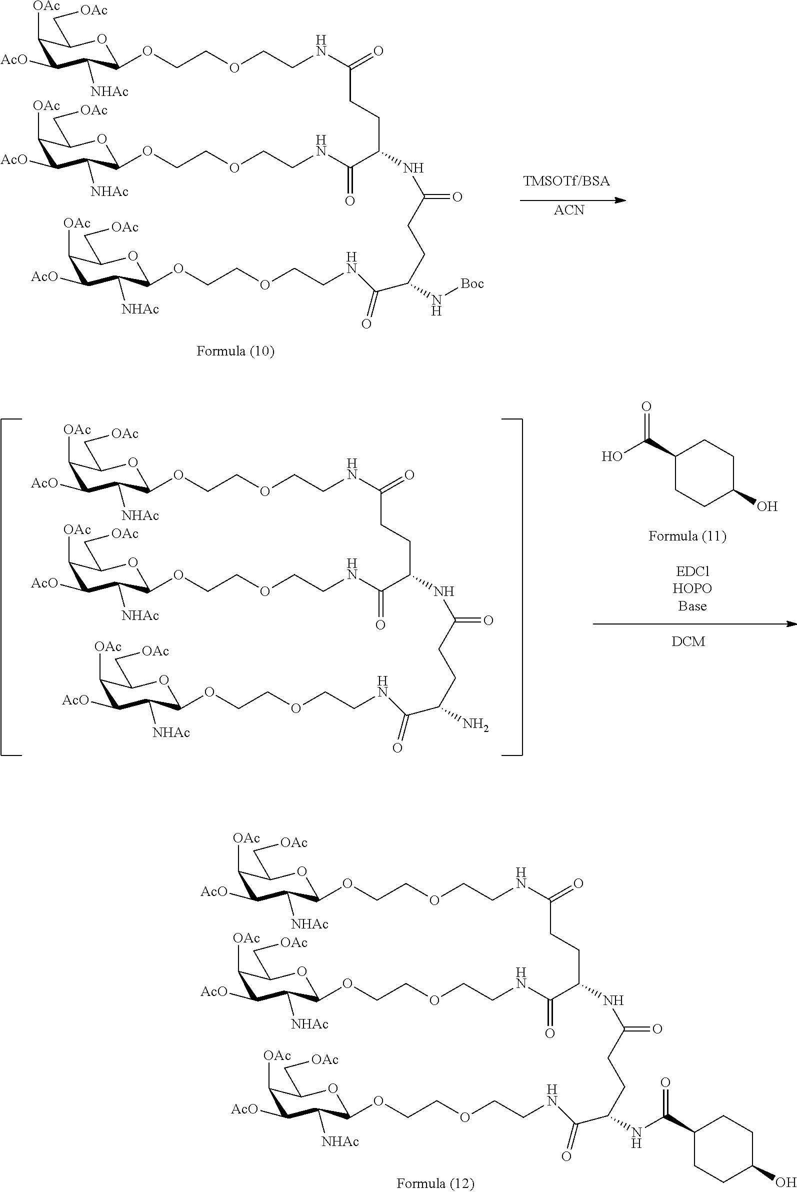

To a reactor under nitrogen at 25-30° C. was charged Formula (10) (180.0 g, 1.0 eq., KF 0.26%) followed by acetonitrile (900 mL, 5 V, KF: 0.02%). The temperature was adjusted to 20-25° C. and BSA (0.50 eq.) was charged into the reactor. The mixture was stirred and TMSOTf (31.0 g, 1.26 eq.) was added dropwise at 20-25° C. The mixture was stirred for 3 h at 20-30° C. to become a solution. The reaction was quenched by addition of water (1.5 eq.) at 20-30° C. and stirred for 15-30 min. Dichloromethane (900 mL, 5 V) was added followed by DIEA (21.5 g, 1.5 eq.) at 20-30° C. The carboxylic acid Formula (11) (17.6 g, 1.1 eq.) was added. The mixture was stirred and cooled to 5-10° C. and HOPO (2.5 g, 0.2 eq.) followed by EDCI (27.6 g, 1.3 eq.) were added to afford a solution that was warmed to 20-30° C. and stirred for 3 hours. Dichloromethane (1800 mL, 10 V) was added, and the mixture was stirred with dibasic 1 M K₂HPO₄ aq. (2700 mL, 15 V) at 20-30° C. for about 30 minutes. (pH=7-8, Assay lost in aqueous: 0.4%). The layers were separated, and the organic layer washed with dibasic 1 M K₂HPO₄ aq. (900 mL, 5 V). (pH=7-8, Assay lost in aqueous: 0.2%) and the layers separated. The organic layer was then washed with monobasic 1 M KH₂PO₄ aq. (5 V). (pH=7-8, Assay lost in aqueous: 2.0%) and the layers separated. The organic layer was concentrated to 5-10 V (KF: 0.64%, DCM/ACN ˜50:50%) and the solution cooled to 0-10° C. MTBE (900 mL, 5 V) was added into the mixture at 0-10° C. at a rate of about 5 mL/min followed by additional MTBE (2700 mL, 15 V) added at a rate of about 30 mL/min at 0-10° C., to afford a suspension that was stirred for 16 h at 20-30° C. The suspension was filtered, and the cake was washed with MTBE (360 mL, 2 V) twice. The wet cake was dried in oven under vacuum at 50-55° C. for 4 h to obtain 162.0 g of Formula (12) as a white solid (81% yield, 97.4 A %).

¹H NMR (400 MHz, DMSO-d₆) δ ppm 1.37-1.45 (m, 4H) 1.78-1.79 (m, 9H) 1.89 (s, 9H) 1.99 (s, 9H) 2.10 (s, 9H) 1.55-2.28 (m, 14H) 3.13-3.25 (m, 6H) 3.31-3.42 (m, 7H) 3.45-3.50 (m, 3H) 3.52-3.61 (m, 4H) 3.74-3.81 (m, 4H) 3.83-3.92 (m, 3H) 4.03 (s, 9H) 4.13-4.21 (m, 2H) 4.24-4.30 (m, 1H) 4.55 (br d, J=8.31 Hz, 3H) 4.98 (dd, J=11.25, 3.18 Hz, 3H) 5.22 (d, J=3.42 Hz, 3H) 7.72-7.85 (m, 6H) 7.90-7.94 (m, 2H)

### Example 7. Preparation of Formula (14)

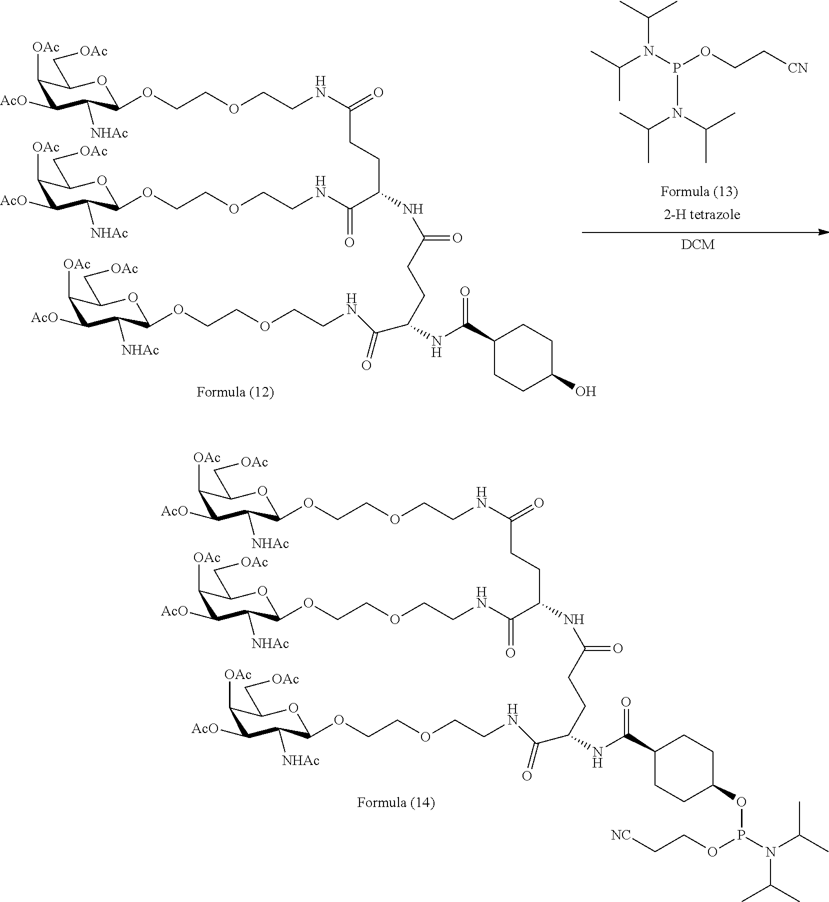

To a reactor under nitrogen at 25-30° C. was charged Formula (12) (100 g, 1.0 eq.) followed by dichloromethane (2000 mL, 20V). The solution was concentrated to 7-9 V by vacuum distillation, keeping batch temperature below 30° C. Dichloromethane (2000 mL, 20 V) was added and the distillation to 7-9 V was repeated. The batch temperature was adjusted to 15-25° C. and the phos reagent Formula (13) (27.4 g, 1.5 eq.) was added. The batch temperature was adjusted further to 0-10° C. and tetrazole (2.5 g, 0.6 eq.) was charged. The reactor walls were washed with dichloromethane (100 mL, 1V) and the mixture warmed to 20-30° C. and stirred for 1-3 hours. The batch temperature was adjusted to 0-10° C. and the reaction quenched by addition of sat. NaHCO₃ aq. (1000 mL, 10 V). The layers were separated, and the organic phase was washed with water (500 mL, 5V) at 0-10° C. Dichloromethane (1000 mL, 10 V) was added to the organic phase and the solution was concentrated to about 9V by vacuum distillation, keeping batch temperature at 0-10° C. Dichloromethane (1000 mL, 10 V) was added to the organic phase and the solution was concentrated to 5-7V by vacuum distillation, keeping batch temperature at 0-10° C.

To a clean reactor was added MTBE (4000 mL, 40 V). The dichloromethane solution was added slowly into the stirring MTBE at 0-10° C. and the resulting suspension was stirred at 0-10° C. for 0.5-2 h and filtered. The cake was washed with MTBE (200 mL, 2 V). Dichloromethane (800 mL, 8 V) was added to the reactor and the temperature adjusted to 5-15° C. The wet solid was added, the mixture stirred, and MTBE (4000 mL, 40 V) was added slowly at 5-15° C. to afford a suspension that was stirred at 5-15° C. for 0.5-2 h and filtered. The cake was washed with MTBE (200 mL, 2 V). The precipitation was repeated, dichloromethane (800 mL, 8 V) was added to the reactor and the temperature adjusted to 5-15° C. The wet solid was added, the mixture stirred, and MTBE (4000 mL, 40 V) was added slowly at 5-15° C. to afford a suspension that was stirred at 5-15° C. for 0.5-2 h and filtered. The cake was washed with MTBE (200 mL, 2 V) and dried under vacuum at 25-35° C. for 24-30 h to afford 85 gram of Formula (14) (75% yield) as a white solid.

¹H NMR (400 MHz, DMSO-d₆) δ ppm 1.15 (dd, J=6.85, 1.22 Hz, 12H) 1.50 (br s, 4H) 1.78-1.79 (m, 9H) 1.90 (s, 9H) 2.00 (s, 9H) 2.11 (s, 9H) 1.61-2.37 (m, 13H) 2.76 (t, J=5.87 Hz, 2H) 3.14-3.26 (m, 6H) 3.34-3.93 (m, 25H) 4.04 (s, 10H) 4.15-4.22 (m, 2H) 4.56 (dd, J=8.44, 2.32 Hz, 3H) 4.97-5.02 (m, 3H) 5.22 (d, J=3.42 Hz, 3H) 7.79-7.86 (m, 6H) 7.91-7.96 (m, 2H). ³¹P NMR (162 MHz, DMSO-d₆) δ ppm 145.01 (s, 1 P).

What is claimed:

1. A method for preparing a poly-N-acetyl-galactosamines (poly-NAG) compound of Formula (X), 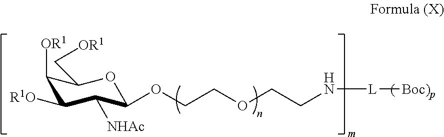
or a salt or stereoisomer thereof, wherein:
R¹ is H or acetyl;
n is an integer from 0 to 4;
m is an integer from 3 to 6;
p is an integer from 1 to 3;
Boc is tert-butyloxycarbonyl and
L is a branched linker comprising (1) m number of 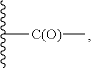
each of the wavy line indicates an attachment point to the remainder of the poly-NAG compound, or a salt or stereoisomer thereof via nitrogen, and (2) p number of —NH—*, each of the (*) indicates an attachment point to the Boc group,
the method comprising coupling a N-acetyl-galactosamine derivative of Formula (X-a), 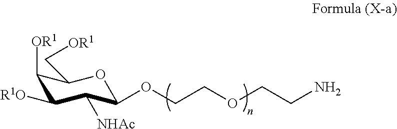
or a salt thereof, to a C₈-C₃₀ compound comprising (1) m number of carboxyl groups or ester groups, and (2) p number of Boc protected primary amine groups, to form the poly-NAG compound, or a salt or stereoisomer thereof, and optionally where the compound of Formula (X-a) has been deprotected in situ in the presence of the C₈-C₃₀ compound, preferably where the compound of Formula (X-a) and the C₈-C₃₀ compound are present in a stoichiometric ratio so a salt forms between X-a and the C₈-C₃₀ compound.

2. The method of claim 1, wherein the poly-NAG compound of Formula (X), or a salt or stereoisomer thereof has a structure of

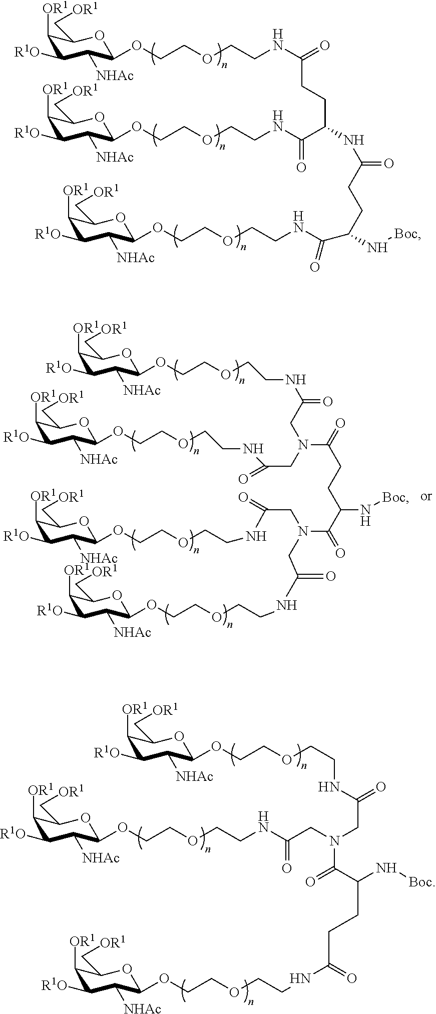

3. The method of claim 2, wherein the poly-NAG compound of Formula (X), or a salt or stereoisomer thereof has a structure of

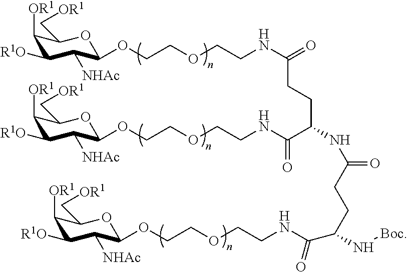

4. The method of claim 1, further comprising purifying the C₈-C₃₀ compound by crystallization prior to coupling to the N-acetyl-galactosamine derivative of Formula (X-a), or a salt thereof.

5. The method of claim 1, wherein the ester groups of the C₈-C₃₀ compound are benzyl (Bn) protected carboxyl groups.

6. The method of claim 4, further comprising deprotecting the Bn groups of the C₈-C₃₀ compound.

7. The method of claim 6, wherein the steps of deprotecting the Bn groups and coupling the N-acetyl-galactosamine derivative of Formula (X-a), or a salt thereof to the C₈-C₃₀ compound are performed together.

8. The method of claim 1, wherein the C₈-C₃₀ compound comprises m number of carboxyl groups or Bn protected carboxyl groups, and one Boc protected primary amine group.

9. The method of claim 8, wherein the C₈-C₃₀ compound is

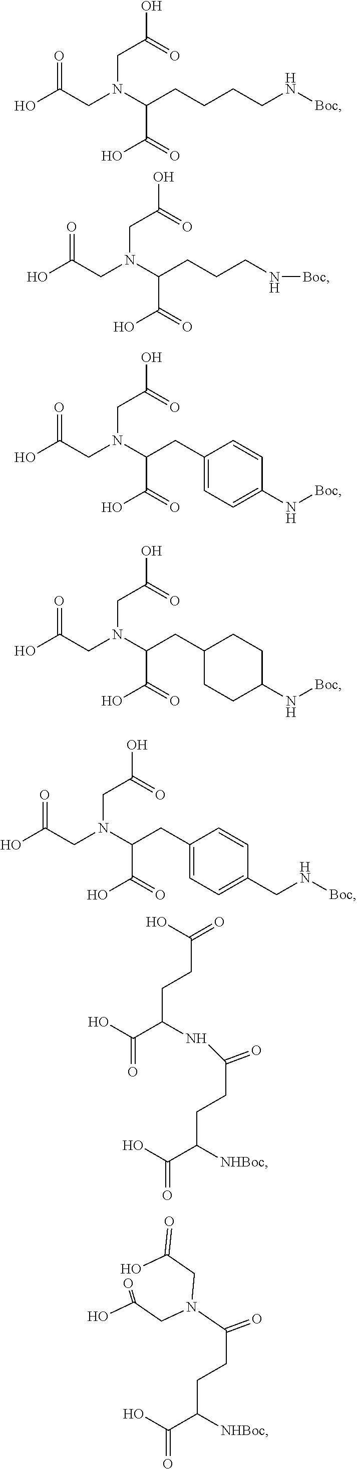

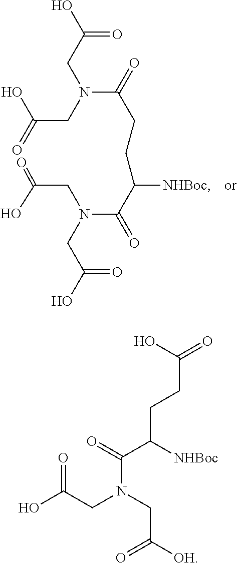 [SMILES: CC(C)(C)OC(=O)NC(CCC(=O)N(CC(=O)O)CC(=O)O)C(=O)N(CC(=O)O)CC(=O)O ; CC(C)(C)OC(=O)NC(CCC(=O)O)C(=O)N(CC(=O)O)CC(=O)O]

10. The method of claim 9, wherein the C₈-C₃₀ compound is

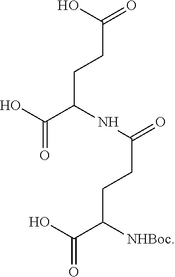 [SMILES: CC(C)(C)OC(=O)NC(CCC(=O)NC(CCC(=O)O)C(=O)O)C(=O)O]

11. The method of claim 8, wherein the C₈-C₃₀ compound is

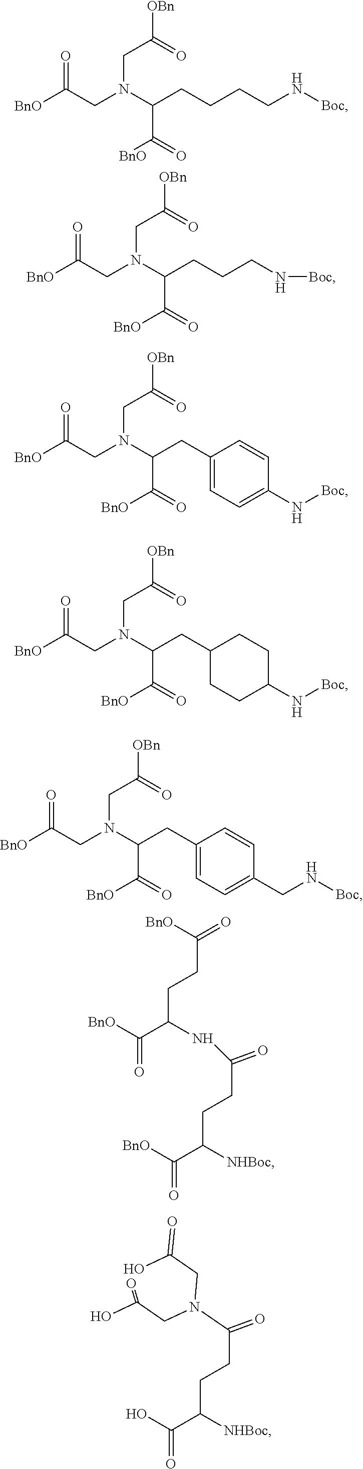

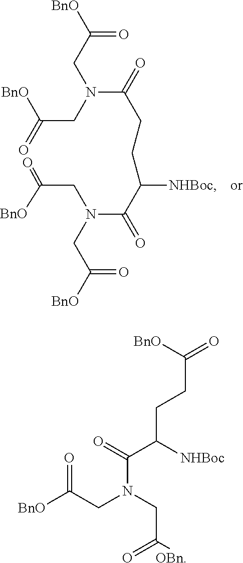 [SMILES: CC(C)(C)OC(=O)NC(CCC(=O)N(CC(=O)OCc1ccccc1)CC(=O)OCc1ccccc1)C(=O)N(CC(=O)OCc1ccccc1)CC(=O)OCc1ccccc1 ; CC(C)(C)OC(=O)NC(CCC(=O)OCc1ccccc1)C(=O)N(CC(=O)OCc1ccccc1)CC(=O)OCc1ccccc1]

12. The method of claim 11, wherein the C₈-C₃₀ compound is

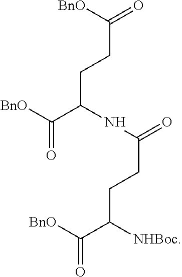 [SMILES: CC(C)(C)OC(=O)NC(CCC(=O)NC(CCC(=O)OCc1ccccc1)C(=O)OCc1ccccc1)C(=O)OCc1ccccc1]

13. The method of claim 1, further comprising deprotecting the Boc group of the poly-NAG compound of Formula (X), or a salt or stereoisomer thereof, to obtain a primary amine compound of Formula (X-b), 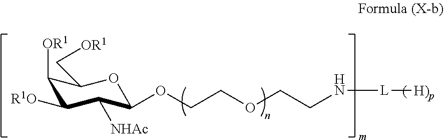
or a salt or stereoisomer thereof.

14. The method of claim 13, wherein the primary amine compound, or a salt or stereoisomer thereof has a structure of 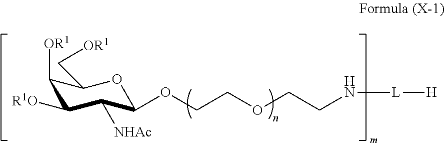
or a salt thereof.

15. The method of claim 13, wherein the primary amine compound, or a salt or stereoisomer thereof has a structure of 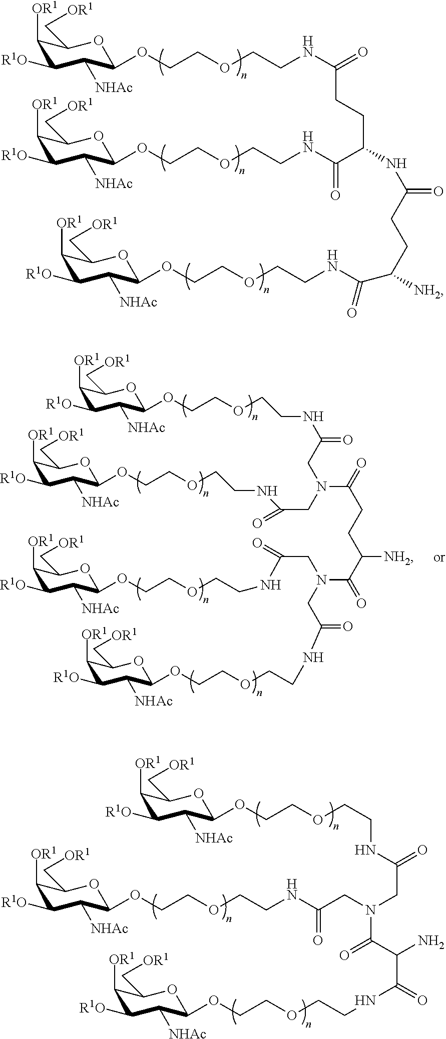
or a salt thereof.

16. The method of claim 15, wherein the primary amine compound, or a stereoisomer thereof has a structure of 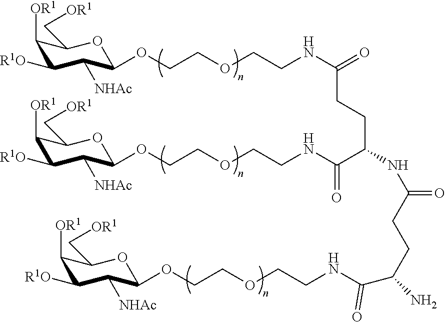
or a salt thereof, preferably a TFA and/or TfOH salt.

17. The method of claim 13, further comprising reacting the primary amine compound, or a salt or stereoisomer thereof, through a condensation reaction with an acid of Formula (X-c), 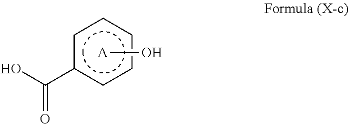
or a stereoisomer thereof, wherein ring A is cyclohexanyl or phenyl,
to obtain a compound of Formula (X-d), 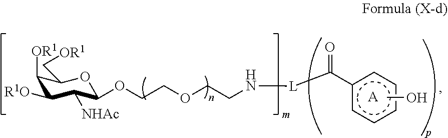
or a salt or stereoisomer thereof.

18. The method of claim 17, wherein the compound of Formula (X-d), or a salt or stereoisomer thereof has a structure of 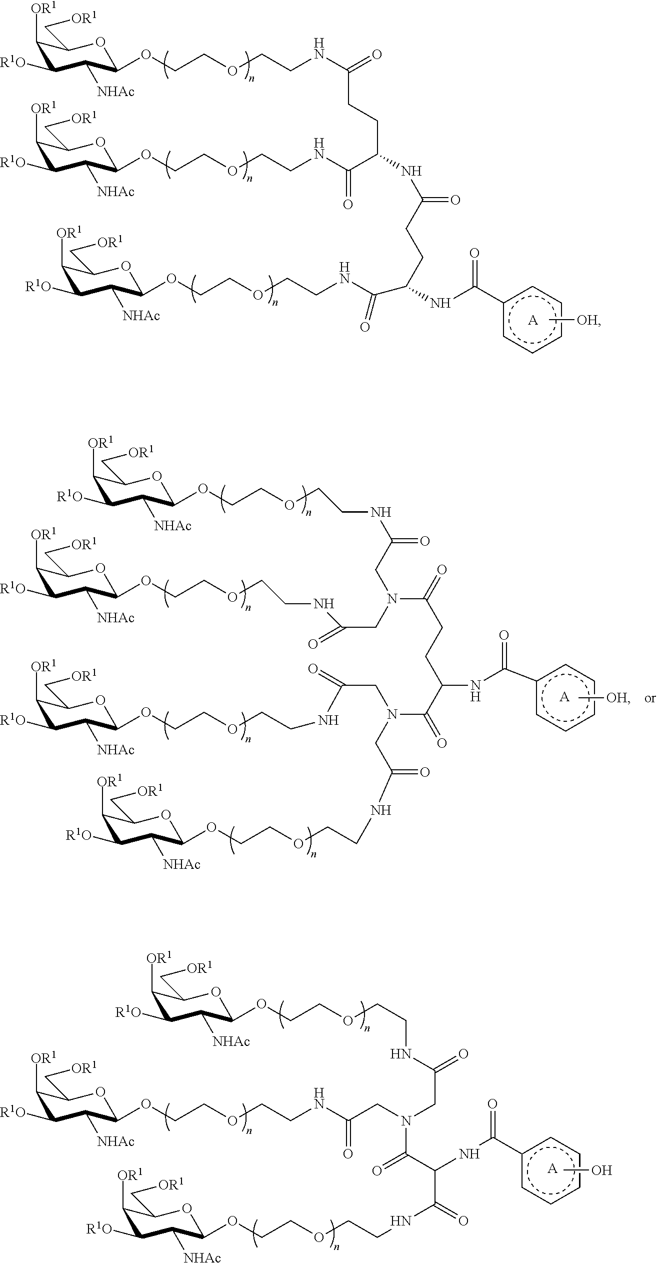
or a salt thereof.

19. The method of claim 17, wherein the compound of Formula (X-d), or a salt or stereoisomer thereof has a structure of: 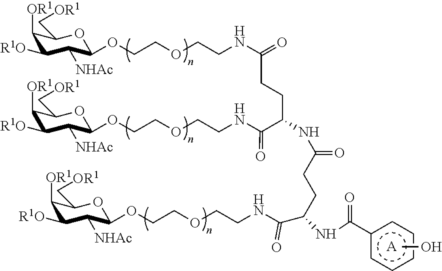
or a salt thereof.

20. The method of claim 19, wherein the compound of Formula (X-d), or a salt or stereoisomer thereof has a structure of 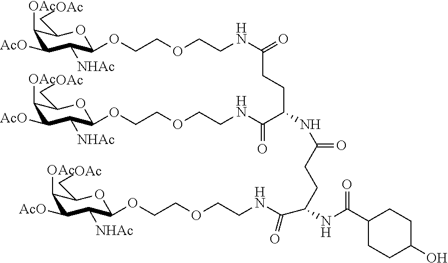
or a salt thereof.

21. The method of claim 17, wherein the steps of deprotecting the Boc group and reacting with the acid of Formula (X-c), or a salt or stereoisomer thereof, are performed together (e.g., without fully isolating a purified intermediate).

22. The method of claim 17, further comprising linking the compound of Formula (X-d), or a stereoisomer thereof to a phosphoramidite reagent through a phosphitylation reaction forming a phosphoramidite compound of Formula (X-e), 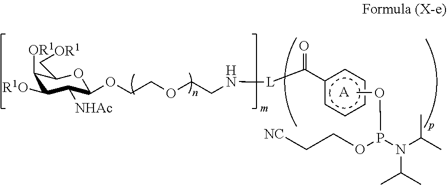
or a salt or stereoisomer thereof.

23. The method of claim 22, wherein the phosphoramidite compound of Formula (X-e), or a salt or stereoisomer thereof has a structure of:

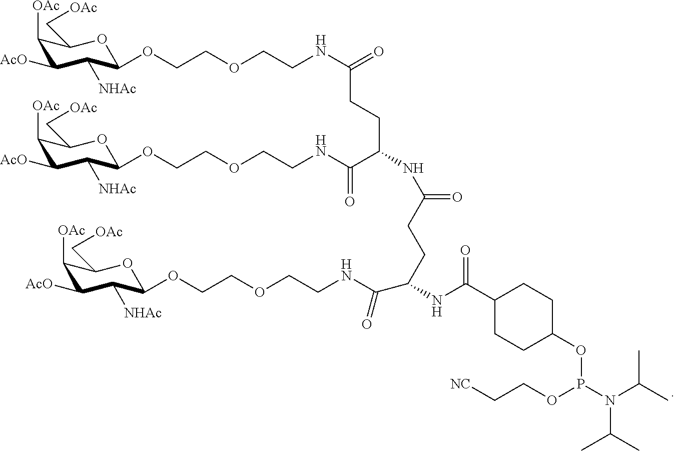

24. The method of claim 23, wherein the phosphoramidite compound of Formula (X-e), or a salt or stereoisomer thereof has a structure of:

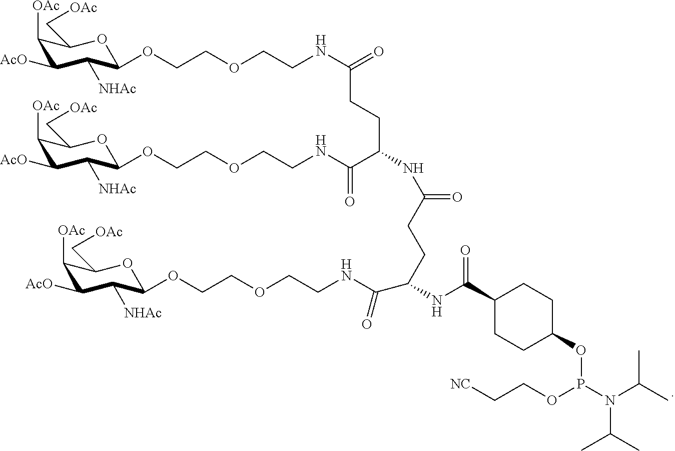

25. The method of claim 22, wherein the phosphoramidite reagent is 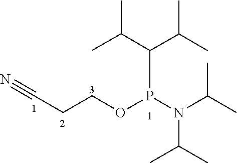 [SMILES: CC(C)C(C(C)C)P(OCCC#N)N(C(C)C)C(C)C]
3-((bis(diisopropylamino)phosphaneyl)oxy)propanenitrile or 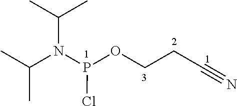
3-((chloro(diisopropylamino)phosphaneyl)oxy)propanenitrile.

26. The method of claim 22, further comprising covalently attaching a therapeutic agent to the phosphoramidite compound of Formula (X-e), or a salt or stereoisomer thereof.

27. The method of claim 26, wherein the therapeutic agent is an expression-inhibiting oligomeric compound.

28. The method of claim 27, wherein the expression-inhibiting oligomeric compound is an RNAi agent.

29. The method of claim 1, wherein the C₈-C₃₀ compound further comprises one or more amide bonds.

30. The method of claim 29, further comprising reacting a first compound comprising one or more carboxyl groups with a second compound comprising one or more primary amine groups via an amide reaction to produce the C₈-C₃₀ compound.

31. The method of claim 30, wherein the ester groups of the C₈-C₃₀ compound are Bn protected carboxyl groups.

32. The method of claim 31, further comprising deprotecting the Bn groups of the C₈-C₃₀ compound.

33. The method of claim 32, wherein the steps of reacting of the first and second compounds, and deprotecting the Bn groups are performed together prior to coupling to the N-acetyl-galactosamine derivative of Formula (X-a), or a salt thereof.

34. The method of claim 1, further comprising deprotecting a benzyloxycarbonyl (Cbz) group of a protected N-acetyl-galactosamine derivative of Formula (X-f), 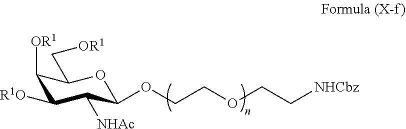
or a salt thereof, to obtain the N-acetyl-galactosamine derivative of Formula (X-a), or a salt thereof, optionally where the protected N-acetyl-galactosamine derivative of Formula (X-f) is deprotected in the presence of the C₈-C₃₀ compound, such as the tri-acid of Formula (4), thereby forming a C₈-C₃₀ compound acid salt of the N-acetyl-galactosamine derivative of Formula (X-a).

35. The method of claim 34, wherein the step of deprotecting the Cbz group is conducted in flow chemistry.

36. The method of claim 34, wherein the salt is a trifluoroacetic acid (TFA) salt.

37. The method of claim 34, wherein the steps of deprotecting the Cbz group, and coupling the N-acetyl-galactosamine derivative of Formula (X-a), or a salt thereof to the C₈-C₃₀ compound are performed together (e.g., without fully isolating a purified intermediate and/or performing the deprotection in the presence of the C₈-C₃₀ compound).

38. The method of claim 34, wherein the ester groups of the C₈-C₃₀ compound are Bn protected carboxyl groups.

39. The method of claim 38, further comprising deprotecting the Bn groups of the C₈-C₃₀ compound.

40. The method of claim 39, wherein the steps of deprotecting the Cbz group, deprotecting the Bn groups, and coupling the N-acetyl-galactosamine derivative of Formula (X-a), or a salt thereof to the C₈-C₃₀ compound are performed together (e.g., without fully isolating a purified intermediate).

41. The method of claim 1, wherein n is 3 or 4.

42. The method of claim 1, wherein p is 1.

43. The method of any one of claims 1-42, wherein R¹ is acetyl and n is 1.

44. A method for preparing a phosphoramidite compound of Formula (I) 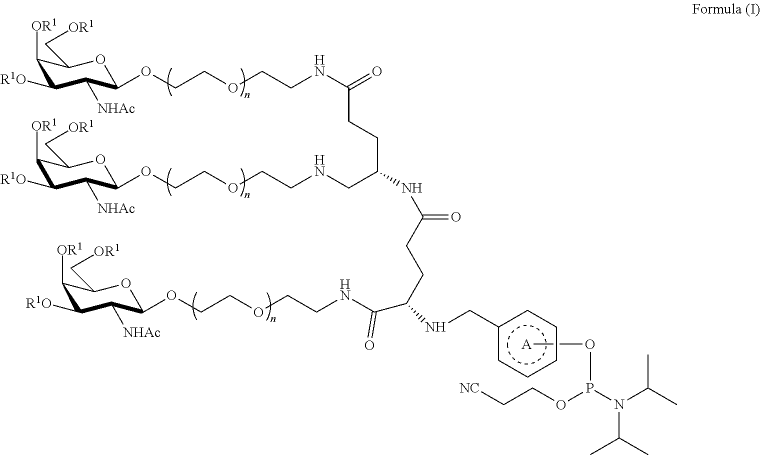
or a stereoisomer thereof, wherein R¹ is H or acetyl, n is an integer from 0 to 4, and ring A is cyclohexanyl or phenyl, comprising:
(i) reacting a compound of Formula (1), 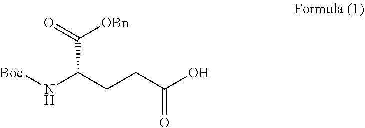 [SMILES: CC(C)(C)OC(=O)N[C@@H](CCC(=O)O)C(=O)OCc1ccccc1]
or a stereoisomer thereof, with a compound of Formula (2), 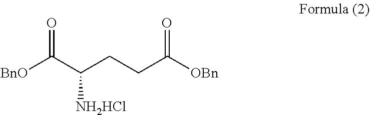
or a stereoisomer thereof, to obtain a compound of Formula (3), 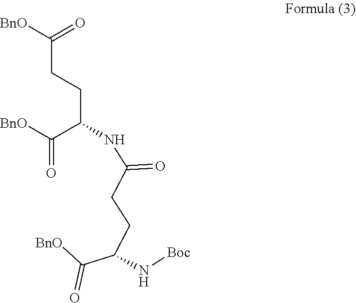 [SMILES: CC(C)(C)OC(=O)N[C@@H](CCC(=O)N[C@@H](CCC(=O)OCc1ccccc1)C(=O)OCc1ccccc1)C(=O)OCc1ccccc1]
or a stereoisomer thereof;
(ii) deprotecting three benzyl (Bn) groups of the compound of Formula (3) or a stereoisomer thereof, to obtain a tri-acid compound of Formula (4), 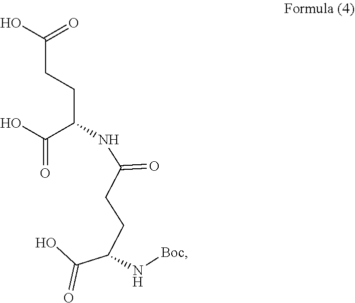 [SMILES: CC(C)(C)OC(=O)N[C@@H](CCC(=O)N[C@@H](CCC(=O)O)C(=O)O)C(=O)O]
or a stereoisomer thereof;
(iii) coupling a N-acetyl-galactosamine derivative of Formula (X-a), or a salt thereof, 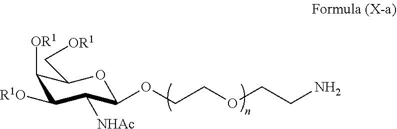
to the tri-acid compound of Formula (4) or a stereoisomer thereof, to form a tri-N-acetyl-galactosamines (tri-NAG) compound of Formula (5), 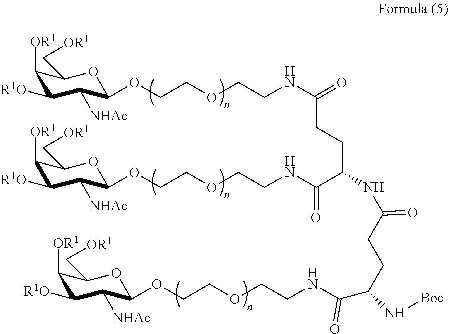
or a stereoisomer thereof;
(iv) deprotecting a tert-butyloxycarbonyl (Boc) group of the tri-N-acetyl-galactosamines (tri-NAG) compound of Formula (5) or a stereoisomer thereof, to obtain a tri-N-acetyl-galactosamines (tri-NAG) compound of Formula (6), 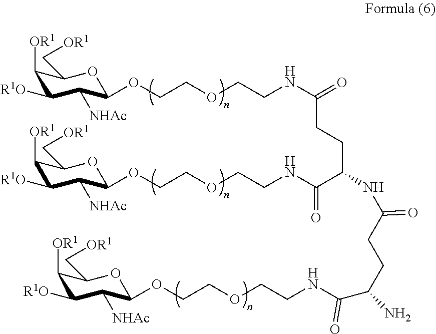
or a stereoisomer thereof;
(v) reacting the tri-N-acetyl-galactosamines (tri-NAG) compound of Formula (6) or a stereoisomer thereof, with an acid of Formula (X-c) 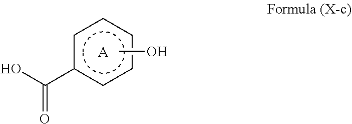
or a stereoisomer thereof, through a condensation reaction between the primary amine group of the tri-N-acetyl-galactosamines (tri-NAG) compound of Formula (6) and the carboxyl group of the acid of Formula (X-c) or a stereoisomer thereof, to obtain a tri-N-acetyl-galactosamines (tri-NAG) compound of Formula (7) or a stereoisomer thereof 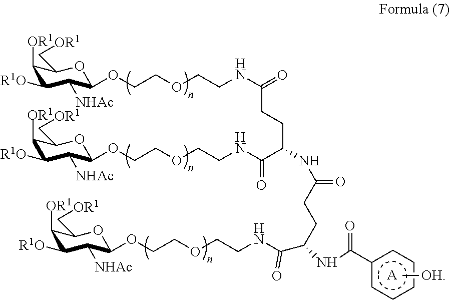
(vi) linking the tri-N-acetyl-galactosamines (tri-NAG) compound of Formula (7) or a stereoisomer thereof, to a phosphorus atom of a phosphoramidite reagent through a phosphitylation reaction forming the phosphoramidite compound of Formula (I) or a stereoisomer thereof.

45. The method of claim 44, wherein the steps (i) and (ii) are performed together.

46. The method of claim 44 or 45, wherein the steps (ii) and (iii) are performed together.

47. The method of any one of claims 44-46, wherein the steps (iv) and (v) are performed together.

48. The method of claim 44, wherein the tri-acid compound of Formula (4), or a stereoisomer thereof, in the step (II) is purified by crystallization.

49. The method of claim 48, wherein the crystallization is performed in a solvent, which is selected from the group consisting of acetonitrile (MeCN), tetrahydrofuran (THF), isopropylacetate (IPAc), water, isopropyl alcohol (IPA), and any combination thereof.

50. The method of claim 44, wherein coupling in step (iii) is performed in the presence of an agent.

51. The method of claim 50, wherein the agent is 1-Ethyl-3-(3-dimethylaminopropyl)carbodiimide (EDCI), 1-2-(1H-benzotriazol-1-yl)-1,1,3,3-tetramethyluronium hexafluorophosphate (HBTU), hydroxybenzotriazole (HOBt) or any combination thereof.

52. The method of claim 51, wherein the agent is EDCI.

53. The method of claim 44, wherein the condensation reaction in the step (v) is conducted in the presence of an agent.

54. The method of claim 53, wherein the agent is selected from 1-Ethyl-3-(3-dimethylaminopropyl)carbodiimide (EDCI), hydroxybenzotriazole (HOBt), 2-Hydroxypyridine-N-oxide (HOPO) or combination thereof.

55. The method of claim 54, wherein the agent is EDCI.

56. The method of claim 44, wherein the phosphitylation reaction in the step (vi) is conducted in the presence of an agent.

57. The method of claim 56, wherein the agent is selected from tetrazole, ethylthiotetrazole (ETT), benzylthiotetrazole (BTT), N-methylimidazole (NMI) or dicyanoimidazole (DCI), and combination thereof.

58. The method of claim 57, wherein the agent is tetrazole.

59. The method of claim 44, wherein the molar ratio of the phosphoramidite reagent to the tri-NAG compound of Formula (7) or a stereoisomer thereof, in the step (vi) is about 1.5.

60. The method of claim 44, wherein the phosphoramidite compound of Formula (I) or a stereoisomer thereof is dried and stored below room temperature.

61. The method of any one of claims 44-60, further comprising deprotecting a benzyloxycarbonyl (Cbz) group of a protected N-acetyl-galactosamine derivative of Formula (8), 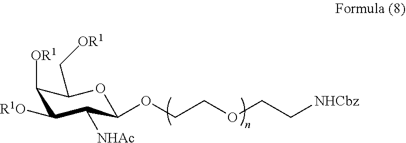
to obtain the N-acetyl-galactosamine derivative of Formula (X-a), or a salt thereof.

62. The method of claim 61, wherein the salt is a trifluoroacetic acid (TFA) salt.

63. The method of claim 61, wherein the step (iii) and the step of deprotecting the benzyloxycarbonyl (Cbz) group are performed together.

64. The method of claim 61, wherein the steps (ii) and (iii), and the step of deprotecting the benzyloxycarbonyl (Cbz) group are performed together.

65. The method of any one of claims 44-64, wherein R¹ is acetyl and n is 1.

66. A phosphoramidite compound of Formula (I), or a stereoisomer thereof prepared by the method of any one of claims 44-65.

67. A therapeutic compound prepared by covalently attaching a therapeutic agent via the phosphorus atom to the phosphoramidite compound of Formula (I) of claim 66, or a stereoisomer thereof.

68. A tri-N-acetyl-galactosamines (tri-NAG) compound having a structure of 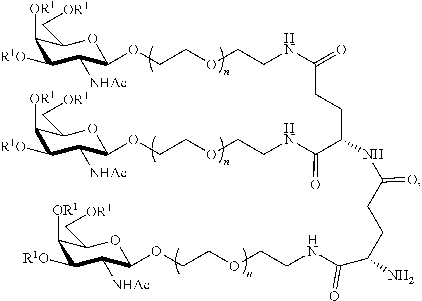
or a stereoisomer thereof, wherein R¹ is H or acetyl, and n is an integer from 0 to 4.

69. A tert-butyloxycarbonyl (Boc) group protected tri-N-acetyl-galactosamines (tri-NAG) compound having a structure of 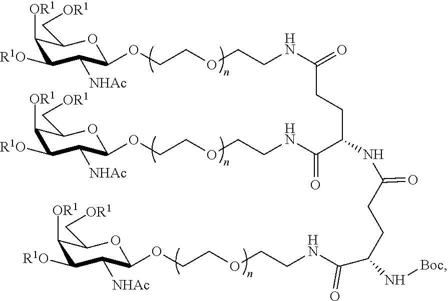
or a stereoisomer thereof, wherein R¹ is H or acetyl, and n is an integer from 0 to 4.

70. An intermediate having a structure of 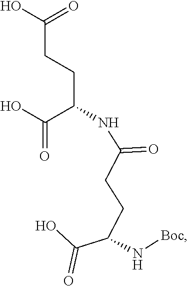 [SMILES: CC(C)(C)OC(=O)N[C@@H](CCC(=O)N[C@@H](CCC(=O)O)C(=O)O)C(=O)O]
or a stereoisomer thereof.

71. An intermediate having a structure of 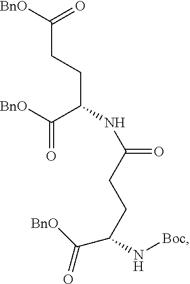 [SMILES: CC(C)(C)OC(=O)N[C@@H](CCC(=O)N[C@@H](CCC(=O)OCc1ccccc1)C(=O)OCc1ccccc1)C(=O)OCc1ccccc1]
or a stereoisomer thereof.

72. The intermediate of claim 70 or 71, wherein the intermediate is in a crystalline form.
